SEQUENCE LISTING (30 sequences)

SEQ ID NO: 1 - DNA, 2185 bp, Homo sapiens
  misc_feature (161)..(161): A,T,G or C
gaattccggctctcaaattttctatagaatcaagatagaacctttagatgtctcaccacgaaactagcaactggaatgaagatagaaacaagtggttataactcagacaaactaatttgtcgagggtttattggaacacctgccccaccggtttgcgacanaagtttctcctgtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttctgggctctatgtttcctttaccccgaattctgtttgccatggcccgggatggcttactgtttagatttcttgccagagtgagtaagaggcagtcaccagttgctgccacgttgactgcaggggtcatttctgctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcacgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagat

SEQ ID NO: 2 - protein, 657 aa, Homo sapiens
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSMFPLPRILFAMARDGLLFRFLARVSKRQSPVAATLTAGVISALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLTLFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 3 - DNA, 2069 bp, Homo sapiens
cgagggtttattggaacacctgccccaccggtttgcgacagcaagtttctcctgtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttcttggatccattttcccaatgcctcgtgtaatctatgctatggcggaggatgggttgcttttcaaatgtctagctcaaatcaattccaaaacgaagacaccaataattgctactttatcatcgggtgcagtggcagctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagat

SEQ ID NO: 4 - protein, 658 aa, Homo sapiens
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSIFPMPRVIYAMAEDGLLFKCLAQINSKTKTPIIATLSSGAVAALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLALFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 5 - DNA, 19 bp, Homo sapiens
ctcctgtcgccttcgtcag

SEQ ID NO: 6 - DNA, 20 bp, Homo sapiens
tcagacatcgggcaaaggtc

SEQ ID NO: 7 - DNA, 23 bp, Homo sapiens
aatgattccttgcagagccgcgc

SEQ ID NO: 8 - DNA, 23 bp, Homo sapiens
cttgccagagtgagtaagaggca

SEQ ID NO: 9 - DNA, 21 bp, Homo sapiens
gccatcaaagcagaaatgacc

SEQ ID NO: 10 - DNA, 24 bp, Homo sapiens
accagttgctgccacgttgactgc

SEQ ID NO: 11 - DNA, 22 bp, Homo sapiens
tgctactttatcatcgggtgca

SEQ ID NO: 12 - DNA, 20 bp, Homo sapiens
atcatgtccacaagcgcctt

SEQ ID NO: 13 - DNA, 26 bp, Homo sapiens
aaacagaaaggccatcaaagctgcca

SEQ ID NO: 14 - DNA, 19 bp, Homo sapiens
gcaattggcttcctgattt

SEQ ID NO: 15 - DNA, 19 bp, Homo sapiens
gcaaacagattggtcagtt

SEQ ID NO: 16 - DNA, 19 bp, Homo sapiens
gtggcaaactggaagatta

SEQ ID NO: 17 - DNA, 19 bp, Homo sapiens
gaattacactggtcttgca

SEQ ID NO: 18 - RNA, 19 nt, Homo sapiens
gcaaaugccaucgugauuu

SEQ ID NO: 19 - RNA, 19 nt, Homo sapiens
gcuaacugauaggugaguu

SEQ ID NO: 20 - RNA, 19 nt, Homo sapiens
gucgcauacucgaacauua

SEQ ID NO: 21 - RNA, 19 nt, Homo sapiens
gauuuagacucgucaugca

SEQ ID NO: 22 - DNA, 22 bp, Homo sapiens
tgctactttatcatcgggtgca

SEQ ID NO: 23 - DNA, 20 bp, Homo sapiens
atcatgtccacaagcgcctt

SEQ ID NO: 24 - DNA, 26 bp, Homo sapiens
aaacagaaaggccatcaaagctgcca

SEQ ID NO: 25 - DNA, 23 bp, Homo sapiens
cttgccagagtgagtaagaggca

SEQ ID NO: 26 - DNA, 21 bp, Homo sapiens
gccatcaaagcagaaatgacc

SEQ ID NO: 27 - DNA, 24 bp, Homo sapiens
accagttgctgccacgttgactgc

SEQ ID NO: 28 - DNA, 19 bp, Homo sapiens
ctcctgtcgccttcgtcag

SEQ ID NO: 29 - DNA, 20 bp, Homo sapiens
tcagacatcgggcaaaggtc

SEQ ID NO: 30 - DNA, 23 bp, Homo sapiens
aatgattccttgcagagccgcgc

(19) United States
(12) Patent Application Publication
(10) Pub. No.: US 2010/0021953 A1
(43) Pub. Date: Jan. 28, 2010

(54) Method for Identifying an Agent that Modulates Arginine Transport in a Chondrocyte

(75) Inventors: Graham Belfield, Leicestershire (GB); Stephen Delaney, Leicestershire (GB); Philip Rawlins, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/997,626

(22) PCT Filed: Jul. 31, 2006
(86) PCT No.: PCT/SE06/00923

§ 371 Date: Feb. 1, 2008

(30) Foreign Application Priority Data
Aug. 3, 2005 (SE) 0501767-8

(51) Int. Cl. C12Q 1/02; G06K 9/00

(52) U.S. Cl. 435/29; 382/133

(57) ABSTRACT

The present invention relates to an assay method for identifying an agent that modulates arginine transport in a chondrocyte comprising the steps of: (a) identifying an agent that modulates the activity and/or expression of CAT-2; and (b) measuring arginine transport in the chondrocyte in the presence or absence of said agent, wherein a difference between: (a) arginine transport in the absence of the agent; and (b) arginine transport in the presence of the agent is indicative that the agent can modulate arginine transport in a chondrocyte. Therapeutic agents that modulate the expression or activity of CAT-2B could be beneficial for the treatment of inflammatory diseases, particularly osteoarthritis. For example, a CAT-2B antagonist may be useful for the treatment of osteoarthritis.

### FIELD OF INVENTION

The present invention relates to an assay method for identifying an agent that modulates arginine transport in a chondrocyte. The present invention also relates to inter alia diagnostic methods, imaging methods, methods for identifying arginine derivatives, complexes, processes, compositions, and agents.

### BACKGROUND TO THE INVENTION

Inflammatory disease—such as Osteoarthritis (OA)—are common, debilitating, costly, and largely incurable diseases. Novel approaches to therapy are clearly required. Diseases—such as OA—are characterised by abnormal functioning of chondrocytes, their terminal differentiation and initiation of osteogenesis within articular cartilage tissue, and breakdown of normal cartilage matrix. Genes whose products are involved in chondrogenesis and osteogenesis starting from the common progenitor cells, genes determining the terminal differentiation of chondrocytes and genes whose products trigger breakdown of the cartilaginous matrix are candidates for therapeutic intervention. In OA, progressive deterioration of the cartilage occurs. Varying degrees of inflammation, involvement of the synovial membrane and pannus formation may also be key factors in the progression of the disease and sensation of pain.

The chondrocyte is the cell responsible for cartilage generation and homeostasis. Dysregulation of cell signalling by exposure to cytokines eg., following injury or due to deterioration, leads to increased catabolism of the cartilage by chondrocytes and reduced anabolism. Chondrocytes are activated by a range of inflammatory stimuli including IL-1α, proIL-1α, IL-1β, TNFα, fibronectin fragments, IL-18 and Retinoic Acid. In turn they release more cytokines (including TNFα and IL-6) and cartilage degrading enzymes (including collagenases (e.g. MMP-1, MMP-8 and MMP-13), gelatinises (including MMP-2), ADAMTSs (aggrecanases), stromelysin, MMP-14; cysteine proteinases, cathepsin, serine proteases and glycosidases). Nitric oxide released from chondrocytes plays a major role in the disease (Ann Rheum. Dis. (1992) 51, 1219-1222; FEBS Letters (1994) 350, 9-12; J. Clin. Invest. (1995) 96, 2357-2363; J. Exp. Med. (1996) 184, 1519-1524; Arth. & Rheum. (1996) 39, 643-647; IDrugs (1998) 1, 321-333). NO is synthesised by iNOS (induced by inflammatory cytokines). For sustained elevated NO production, increased arginine is required (Br J Pharmacol. (1995) 116, 3243-50; J. Biol. Chem. (2001) 276, 15881-15885). In other cells, CAT-2 has been described as the inducible arginine transporter for iNOS (J. Biol. Chem (2001) 276, 15881-15885; Eur. J. Physiol. (2004) 447, 532-542).

CATs as targets for the manipulation of NOS activity have been reviewed in Pharm. Biotechnol. (1999) 12, 229-249. It is postulated that the indirect inhibition of NOS activity could be mediated by CAT (including CAT-2B) inhibitory compounds that do not interfere with iNOS directly but inhibit substrate availability. Moreover, compounds that do inhibit NOS may also have an indirect effect on NOS activity through inhibiting substrate availability. The effects of inhibitors are described against Xenopus oocytes injected with human CAT-2B RNA. No CAT-2B specific inhibitors are described.

Clinical Science (2002) 102, 645-650 describes that the increased arginine transport in peripheral blood mononuclear cells from patients with septic shock is consistent with an increase in the expression of CAT-2. It is hypothesised that inhibition of CAT-2 in humans may lead to a successful treatment for septic shock. Also, since increased production of NO is thought to be important in the pathogenesis of ulcerative colitis, rheumatoid arthritis and chronic pain then it is believed that the findings have implications for other diseases.

WO 00/44766 teaches that CAT-2 is involved in L-arginine transport and nitric oxide synthesis in various cell types and pathological conditions. Antisense oligonucleotides directed against CAT-2 mRNA are described for inhibiting cationic amino acid transport. Methods of treating diseases characterised by an undesirable level of nitric oxide are described. Specifically, sepsis, cachexia, neoplastic diseases—such as Kaposi's sarcoma, breast and lung cancer—cerebral malaria, capillary leak syndrome and autoimmune disease including systemic lupus erythematosus, rheumatoid arthritis and multiple sclerosis are disclosed. A method of screening for CAT2A or CAT2B inhibitors is also described.

WO 03/073990 discusses use of a compound that decreases the production of a protein involved in arginine metabolism in the preparation of a medicament for treating asthma or allergies. Said protein may be CAT2, arginase I or arginase II. It also discusses a method of discovering a compound that is effective for treating asthma or allergies that includes: providing a candidate compound; determining whether the compound inhibits arginine metabolism, wherein inhibition of arginine metabolism is indicative that the compound is effective for treating asthma or allergies. Said compound may be an inhibitor of CAT2, arginase I or arginase II. WO 03/073990 further discusses a therapeutic composition for the treatment of asthma or allergies that includes an arginase inhibitor in a pharmaceutically acceptable carrier, for example an inhibitor of CAT2 activity in a pharmaceutically acceptable carrier.

There is a need in the art for improved methods to treat inflammatory diseases.

### SUMMARY OF THE INVENTION

The present invention is based, in part, on the surprising finding that CAT-2 is an arginine transporter in chondroytes. CAT therefore provides arginine to chondrocytes which is used for iNOS and NO production in disease. In particular, CAT-2 plays an important role in iNOS mediated NO production from human primary chondrocytes that are stimulated with a range of chondrocyte activators associated with arthritic diseases—such as Osteoarthritis (OA) and Rheumatoid arthritis (RA). Advantageously, modulating CAT-2 offers a novel means of regulating stimulated chondrocyte iNOS substrate delivery, and as such may be exploited as a mechanism to slow disease modification, reduce inflammation and attenuate associated pain. Accordingly, the present invention finds utility in the treatment of inflammation—such as iNOS mediated inflammation—and more specifically in the treatment of disease—such as arthritic diseases, for example, OA and RA. Since CAT-2 knock-out mice are still viable then a CAT-2 inhibitor would not be expected to give adverse side-effects (J. Biol. Chem. (2001) 276, 15881-15).

We have shown that the arginine transporter CAT-2B (SLC7A2) is essential for Nitric Oxide (NO) production from stimulated osteoarthritis (OA) primary chondrocytes. On treatment of OA chondrocytes with chondrocyte activators, we saw a very large increase in RNA expression for CAT-2B (around 50 fold) and iNOS (around 1000 fold), and a concomitant increase in arginine transport (around 10 fold) and the “switching on” of NO production. Following treatment of cells with CAT-2B-specific siRNA we observed an 80-90% reduction in both CAT-2B RNA expression and arginine transport, and around a 55% reduction in chondrocyte NO production, whilst the expression of iNOS and other arginine transporters were unaffected. Chondrocyte NO release is an important component of the patho-physiology of OA. Therefore, the provision of therapeutic agents that modulate the expression or activity of CAT-2B could be beneficial for the treatment of inflammatory diseases, particularly osteoarthritis. For example, a CAT-2B antagonist may be useful for the treatment of osteoarthritis.

### SUMMARY ASPECTS OF THE PRESENT INVENTION

In a first aspect the present invention relates to an assay method for identifying an agent that modulates arginine transport in a chondrocyte comprising the steps of: (a) identifying an agent that modulates the activity and/or expression of CAT-2; and (b) measuring arginine transport in the chondrocyte in the presence or absence of said agent, wherein a difference between: (a) arginine transport in the absence of the agent; and (b) arginine transport in the presence of the agent is indicative that the agent can modulate arginine transport in a chondrocyte.

Agents that modulate the activity and/or expression of CAT-2 may be identified, for example, by a membrane potential assay or by an arginine transport assay in recombinant cell lines expressing CAT-2B (HEK-293 cell lines).

In a second aspect, there is provided a method of diagnosing a disease or syndrome in a subject comprising the steps of: (a) detecting the level or pattern of expression and/or activity of CAT-2 in a chondrocyte; and (b) comparing the level or pattern of expression and/or activity of CAT-2 with that of a control, wherein a difference between the level or pattern of expression or activity of CAT-2 in the sample comprising the chondrocyte and the control is indicative of the presence of a disease or syndrome.

In a third aspect, a method of diagnosing a disease or syndrome in a subject is provided comprising the steps of: (a) detecting the level and/or pattern of transport of arginine or a derivative thereof in a chondrocyte; and (b) comparing the level and/or pattern of transport of arginine or a derivative thereof with that of an control, wherein a difference between the level and/or pattern of transport of arginine or a derivative thereof in the sample comprising the chondrocyte and the control is indicative of a disease or syndrome.

In a fourth aspect, the present invention relates to a method of imaging a chondrocyte comprising the steps of: (a) providing for the uptake of labelled arginine or a derivative thereof into the chondrocyte; and (b) imaging the chondrocyte.

In a fifth aspect, a method of identifying a derivative of arginine that can be transported into and/or out of a chondrocyte by CAT-2 is provided comprising the steps of: (a) contacting the derivative of arginine with the chondrocyte; and (b) measuring the transport of the derivative of arginine into and/or out of the chondrocyte.

In a sixth aspect, there is provided a complex comprising CAT-2 and arginine in a chondrocyte.

In a seventh aspect, the present invention relates to a process comprising the steps of: (a) performing the assay method according to the first aspect of the present invention; (b) identifying one or more agents that do affect CAT-2 expression and/or activity in a chondrocyte; and (c) preparing a quantity of those one or more identified agents.

In an eight aspect, there is provided an agent obtained or obtainable by the assay method according to the first aspect of the present invention.

In a ninth aspect, the present invention relates to a pharmaceutical composition comprising an agent identified by the assay method according to the first aspect of the present invention or the process according to the seventh aspect of the present invention admixed with a pharmaceutically acceptable carrier, diluent, excipient or adjuvant and/or combinations thereof.

In a tenth aspect, the present invention provides a vaccine composition comprising an agent according to according to the eighth aspect of the present invention.

In an eleventh aspect, the present invention provides a process of preparing a pharmaceutical composition comprising admixing an agent identified by the assay method according to the first aspect of the present invention or the process according to the seventh aspect of the present invention admixed with a pharmaceutically acceptable diluent, carrier, excipient or adjuvant and/or combinations thereof.

In a twelfth aspect, the present invention relates to a method of preventing and/or treating a disease comprising administering an agent according to the eighth aspect of the present invention or a pharmaceutical composition according to the ninth aspect of the present invention or a vaccine according to the tenth aspect, wherein said agent or pharmaceutical composition or vaccine is capable of modulating CAT-2 expression and/or activity in a chondrocyte.

In a thirteenth aspect, there is provided an agent according to the eighth aspect of the present invention or a pharmaceutical composition according to the ninth aspect of the present invention for use in modulating CAT-2 expression and/or activity in a chondrocyte to cause a beneficial preventative and/or therapeutic effect.

In a fourteenth aspect, there is provided the use of an agent according to the eighth aspect of the present invention or a pharmaceutical composition according to the ninth aspect of the present invention in the manufacture of a composition for modulating CAT-2 expression and/or activity in a chondrocyte to cause a beneficial preventative and/or therapeutic effect.

In a fifteenth aspect, the present invention relates to a method of preventing and/or treating a disease comprising administering an agent according to the eighth aspect of the present invention or a pharmaceutical composition according to the ninth aspect of the present invention or a vaccine according to the tenth aspect, wherein said agent or pharmaceutical composition or vaccine is capable of modulating CAT-2 expression and/or activity in a chondrocyte to cause a beneficial preventative and/or therapeutic effect.

In a sixteenth aspect, there is provided the use of one or more of the sequences set forth in Table 2 in the manufacture of a composition for the treatment of an inflammatory disease.

### PREFERRED EMBODIMENTS

Preferably, the chondrocyte is selected from the group consisting of a human primary chondrocyte, a passaged primary chondrocyte, an immortalised chondrocyte derived cell line and a synovial fibroblasts.

Preferably, the chondrocyte is isolated or isolatable from an Osteoarthritis (OA) or Rheumatoid Arthritis (RA) patient.

Preferably, the chrondrocyte is stimulated. More preferably, the chrondrocyte is stimulated by an inflammatory stimulus.

Preferably, the inflammatory stimulus is selected from the group consisting of IL-1α, proIL-1α, IL-1β, TNFα, fibronectin fragment IL-18 and Retinoic Acid.

Preferably, CAT-2 is a CAT-2 isoform. More preferably, the CAT-2 isoform is or is derived from CAT-2A or CAT-2B.

Preferably, the CAT-2 isoform is expressed in a recombinant cell line.

Preferably, the cell line is HEK-293 or CHO-K1.

Preferably, the disease or syndrome is an inflammatory disease. More preferably, the inflammatory disease is an arthritic disease. Most preferably, the disease is OA or RA.

Preferably, arginine transport is detected using a nuclear imaging technique.

Preferably, the nuclear imaging technique is Positron Emission Tomography (PET) or Single Photon Emission Computed Tomography.

### DESCRIPTION OF THE FIGURES

FIG. 1 is a series of graphs showing the expression of CAT2 isoforms in tissues. RNA isolated from a number of tissues by standard methods was subjected to Taqman quantitative rtPCR by standard procedures using primers/probes specific for CAT-2A, CAT-2B and generic for CAT-2 (ie. do not distinguish between CAT-2A and CAT-2B). FIG. 1(a) represents CAT-2 (total) RNA; FIG. 1(b) represents CAT-2A RNA; FIG. 1(c) represents CAT-2B RNA.

FIG. 2 is a graph showing the expression of CAT-2B in cell lines. RNA isolated from a number of tissues and cells by standard methods was subjected to Taqman quantitative PCR using primers/probes specific for CAT-2B.

FIG. 3 is a series of graphs showing the inducible expression of CAT-2 isoforms in OA primary chondrocytes stimulated with IL-1β. RNA isolated from primary chondrocytes (taken from the joints of two OA patents) stimulated with a range of IL-1β concentrations was subjected to Taqman quantitative PCR by standard procedures using primers/probes for (a) CAT-2 (total), CAT-2A and CAT-2B induction with IL-1β; (b) CAT-2 induction with IL-1β in multiple OA donors. Data expressed as relative increase in expression (where 1 ng/ml=100%).

FIG. 4 is a series of graphs showing the inducible expression of CAT-2B in OA chondrocytes stimulated with a range of chondrocyte activators: (a) NO production. (b) Taqman analysis of iNOS RNA expression. (c) CAT-2B RNA. RNA isolated from primary chondrocytes (taken from the joints of two OA patents) stimulated with a range of concentrations of four chondrocyte activators (IL-1β; TNFα; IL-18 & Fibronectin fragments) concentrations was subjected to Taqman quantitative PCR by standard procedures using primers/probes specific for the CAT-2B isoform.

FIG. 5 is a series of graphs showing the expression of iNOS and arginine transporters in OA chondrocytes stimulated with a range of chondrocyte activators. RNA isolated from primary chondrocytes (taken from the joints of an OA patients stimulated with a range of concentrations of four chondrocyte activators (IL-1β; TNFα; IL-18 & Fibronectin fragments) concentrations was subjected to Taqman quantitative PCR using primers/probes specific for iNOS and a range of arginine transporters, specifically CAT-2B, CAT-1, Y⁺LAT1, B^{(0,+)}AT and ATB(0,+). (a) CAT-1 (low expression CT ˜32); (b) CAT-2B (high induction 30-24); (c) y+LAT1 (marginal expression CT ˜34); (d) B0+AT (marginal expression CT ˜36); (e) INOS (high induction CAT 34-24).

FIG. 6 is graph illustrating the co-ordinate CAT-2B and iNOS expression in OA and Post-Mortem Cartilage. iNOS and CAT-2B expression in RNA isolated from articular-cartilage taken from knees of six OA patients and five Post-Mortem Cadavers was analysed.

FIG. 7 is a series of graphs showing the co-ordinate induction of CAT-2B and iNOS in IL-1β stimulated OA primary chondrocytes, with Arg transport and NO readouts Primary chondrocytes (taken from the joints of two OA patents) stimulated with 100 pg/ml IL-1β and subsequent determination of (a) CAT-2B RNA expression (Taqman); (b) iNOS RNA expression (Taqman); (c) Arginine uptake (radioactive 3H arginine uptake assay) and (d) Nitrite production (Griess Assay) are shown.

FIG. 8a is a series of graphs showing the optimisation of the window of siRNA delivery to OA chondrocytes to obtain maximal mRNA knock-down without lipid-mediated loss of chondrocyte viability.

FIG. 8b is a series of graphs showing the results of experiments where the effects and or levels of CAT-2 siRNA and controls (CAT-2 MMsiRNA, lamin and luciferase) were examined in IL-1β stimulated chondrocytes from 2 donors, with the multiple readouts of CAT-2 RNA, NO production and arginine transport.

FIG. 9 is a series of photographs in which immuno chemistry with human anti-CAT2 antibody (Orbigen) demonstrates plasma membrane localised expression of CAT-2 in HEK-293 transfected with pGEN-IRES-NEO[CAT-2B] (a); whereas no CAT-2 expression can be detected in “empty vector” HEK-293 cells transfected with pGEN-IRES-NEO (b).

### DETAILED DESCRIPTION OF THE INVENTION

### Cat

Arginine transport in most cells and tissues is mediated by a transport system encoded by three genes: Cat1, Cat2 and Cat3. Cat1 and Cat2 encode similar proteins, CAT1 and CAT2 (e.g. MacLeod, C. L., Biochem. Soc. Trans., 24: 846-852 (1996)), comprise a transport system, which facilitates the transport of the cationic amino acids lysine, arginine and ornithine in a sodium-independent manner.

Cat2 encodes two protein isoforms (as a result of alternate splicing), CAT-2A and CAT-2B. The CAT-2A protein exhibits a significantly lower (10-fold) apparent affinity for its substrate than either CAT1 or CAT2.

For some aspects of the present invention, the preferred CAT-2 sequence is a CAT-2 isoform sequence—such as CAT-2A or CAT-2B.

The nucleotide sequence of CAT-2A is available in databases (EMBL:HSU76368) and may comprise the sequence set forth in SEQ ID No. 1.

The amino acid sequence of CAT-2A is available in databases (EMBL:HSU76368) and may comprise the sequence set forth in SEQ ID No. 2.

For some aspects of the present invention, the preferred CAT-2 isoform sequence is CAT-2B.

The nucleotide sequence of CAT-2B is available in databases (EMBL:BC069648) and may comprise the sequence set forth in SEQ ID No. 3.

The amino acid sequence of CAT-2B is available in databases (EMBL:BC069648) and may comprise the sequence set forth in SEQ ID No. 4.

### Chondrocyte

As used herein, the term “chondrocyte” refers to the art-recognised use of the term for a cell type involved in cartilage formation and repair. The chondrocyte functions to produce extracellular matrix of proteoglycans and collagen. Also included within the use of this term are chondrocyte precursor cells—such as chondroblasts, mesenchymal stem cells and hypertrophic chondrocytes.

From least to terminally differentiated, the chondrocytic lineage is colony-forming unit-fibroblast (CFU-F), mesenchymal stem cell/marrow stromal cell (MSC), chondrocyte and hypertrophic chondrocyte. When referring to bone or cartilage, mesenchymal stem cells (MSC) are commonly known as osteochondrogenic (or osteogenic, chondrogenic, osteoprogenitor, etc.) cells since a single MSC has shown the ability to differentiate into chondrocytes or osteoblasts, depending on the medium. In vivo, differentiation of a MSC in a vascularized area (such as bone) yields osteoblasts while differentiation of a MSC in a non-vascularized area (such as cartilage) yields a chondrocyte. Chondrocytes undergo terminal differentiation when they become hypertrophic during endochondral ossification. This last stage is characterised by major phenotypic changes in the cell.

Chondrocytes may be isolated from a variety of sources. For example, embryonic chondrocytes can be isolated from sterna (Leboy et al., (1989) J. Biol. Chem., 264:17281-17286) and vertebra (Lian et al., (1993) J. Cellular Biochem., 52:206-219), limb bud mesenchymal cells in micromass cultures (Roark et al., (1994) Develop. Dynam., 200:103-116), growth plate chondrocytes in monolayer (Rosselot et al., (1994) J. Bone Miner. Res., 9:431-439), or pellet cultures (Kato et al., (1988) Proc. Nat. Acad. Sci., 85:9552-9556) have been used to characterise chondrocyte responses to exogenous factors, many of which function in an autocrine manner.

Preferably, the chondrocyte is a chondrocyte from an animal. More preferably, the chondrocyte is a chondrocyte from a human.

Preferably, the chondrocyte is a primary chondrocyte, more preferably, a human primary chondrocyte.

Chondrocytes may be obtained by, for example, a biopsy of cartilaginous tissue from an area treated with local anaesthetic with a small amount of lidocaine injected subcutaneously. The chondrocyte cells from the biopsy tissue can be expanded in culture. The biopsy may be obtained using a biopsy needle, a rapid action needle that makes the procedure quick and simple.

Chondrocytes may be isolated from donor tissue supplied by large cell banks.

The chondrocyte may be isolated from a subject that is suffering or suspected to be suffering from Osteoarthritis (OA) or Rheumatoid Arthritis (RA).

The chondrocyte may be isolated from cartilage.

Preferably, the chondrocyte is from a human primary chondrocyte, a passaged primary chondrocyte (eg. a passaged human primary chondrocyte), an immortalised chondrocyte derived cell line or a synovial fibroblast.

Preferably, the chondrocyte is or is derived from tissues and cells from arthritic joints.

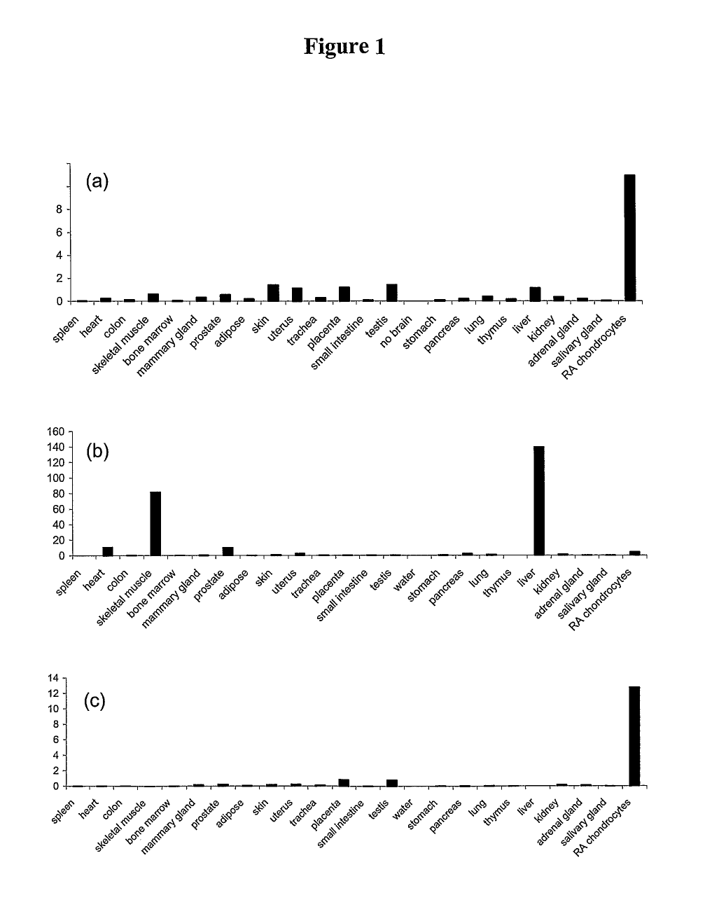

Chondrocyte cells obtained by biopsy may be cultured and passaged as necessary. For example, articular cartilage can be aseptically obtained from calf knee joints. Cartilage can be shaved from the end of the bones using a scalpel. The tissue can be finely minced in saline solution and submitted to collagenase and trypsin digestion until the tissue has completely digested and single cells are generated. The chondrocyte cells isolated from the cartilage tissue may then be washed from enzyme with DMEM containing fetal bovine serum, and seeded onto tissue culture plastic dishes using, for example, about 5,000 cells to about 10,000 cells per square centimeter. The chondrocyte cells can be cultured in DMEM and fetal calf serum with L-glutamine, penicillin and streptomycin at 37° C. in 5% CO2 with an atmosphere having 100% humidity and expanded in number (See FIG. 1). The chondrocyte cells can then be removed from the tissue culture vessel using trypsin/EDTA and passaged into larger tissue culture vessels or encapsulated in gel suspension. Expanded chondrocyte cells can also be frozen according to standard procedures until needed.

Other sources from which chondrocytes may be derived include adipose-derived cells, mesenchymal stem cells, fetal stem cells, marrow-derived stem cells, and other pluripotent stem cells. The chondrocytes may be autogeneic, isogeneic (e.g., from an identical twin), allogeneic, or xenogeneic.

A number of studies have been conducted on the isolated chondrocytes, from which has emerged the critical role for a number of growth factors, including basic fibroblast growth factor (bFGF), transforming growth factor beta (TGF beta), insulin-like growth factor-1 (IGF-1), and parathyroid hormone (PTH), which regulate chondrocyte proliferation and differentiation. The expression of these factors and their associated receptors are maturation dependent and exquisitely regulated (Bohme, et al., (1992) Prog. Growth Factor Res. 4:45-68). Other studies have shown that vitamins A, C, and D are also required for chondrocyte maturation (Leboy et al., (1994) Microscopy Res. and Technique 28:483-491; Iwamoto et al., (1993) Exp. Cell Res., 207:413-420; Iwamoto et al., (1993) Exp. Cell Res. 205:213-224; Pacifici et al., (1991) Exp. Cell Res. 195:38-46; Shapiro et al., (1994) J. Bone Min. Res. 9:1229-1237; Corvol et al. (1980) FEBS Lett. 116:273-276; Gerstenfeld et al., (1990) Conn. Tiss. Res. 24:29-39; Schwartz et al., (1989) J. Bone Miner. Res. 4:199-207; and Suda, (1985) Calcif Tissue Int., 37:82-90).

Chondrocytes may be stimulated by, for example, an inflammatory stimulus, which causes the release of more cytokines and cartilage degrading enzymes. Preferably, the inflammatory stimuli are selected from the group consisting of IL-1α, proIL-1α, IL-1β, TNFα, fibronectin fragments, IL-18 and Retinoic Acid.

Chondrocyte cells may be engineered to incorporate a nucleic acid encoding one or more therapeutic agents—such as proteins, antibodies, regulators and cytokines. Other molecules, genes, or nucleic acids that influence cell growth, matrix production, or other cellular functions such as cell cycle may also be used. In one embodiment, the nucleic acid can be introduced into the chondrocytes as naked DNA, nanocapsules, microspheres, beads, and/or lipid-based systems—such as liposomes—or carriers—such as keyhole limpet hemocyanin (KLH) and human serum albumin. In this regard, reference can be made to U.S. Pat. No. 6,303,379.

In another embodiment, the chondrocytes may be transfected using vectors engineered to carry a nucleic acid that encodes the therapeutic agent. By way of example, the nucleic acid sequences may be cloned into the vector using standard cloning procedures known in the art, as described by Maniatis et al., Molecular Cloning: A Laboratory Manual, Cold Springs Laboratory, Cold Springs Harbor, N.Y. (1982). Suitable vectors include, but are not limited to plasmid vectors—such as pRO-EX (Gibco/BRL), pBR322, pBR325, pACYC177, pACYC184, pUC8, pUC9, pUC18, pUC19, pLG339, pR290, pKC37, pKC101, SV 40, pBluescript II SK<+> or KS<+> (See Stratagene Cloning Systems Catalog from Stratagene, La Jolla, Calif.), pQE, pIH821, pGEX, pET series (See Studier et al., Use of T7 RNA Polymerase to Direct Expression of Cloned Genes, Gene Expression Technology vol. 185 (1990)) and any derivatives thereof.

A genetically modified chondrocyte may be implanted in a subject to treat a disease—such as an inflammatory disease. For example, chondrocytes may be implanted either in or close to a joint capsule for the treatment of specific joint that is affected by an inflammatory disease, or in an ectopic site for a systemic delivery to all joints of an anti-inflammatory therapeutic agent. The genetically modified chondrocyte may comprise or express an agent—such as an antagonist or an agonist—that modulates CAT-2 activity and/or expression as described herein.

In a further aspect, there is provided a complex comprising CAT-2 (eg. CAT-2A and CAT-2B) and arginine in a chondrocyte. Accordingly, there is provided a CAT-2 protein complexed, associated or bound to arginine in a chondrocyte.

### Sample

The term “sample” as used herein, has its natural meaning. The sample may be or may be derived from biological material. The biological material may be in the form of cells, tissues, sections, or extracts of such tissues—such as cells and tissues from cartilage.

The sample may be any physical entity comprising or consisting of chondrocytes.

Cartilage samples may be removed from a subject—such as joint of a subject—by a variety of excision methods.

For some embodiments of the present invention, the sample may be isolated or from a subject that is suffering or suspected to be suffering from an inflammatory disease—such as Osteoarthritis (OA) or Rheumatoid Arthritis (RA).

The cartilage sample may be used as is, or treated with an enzyme—such as collagenase—to break down the cartilage matrix and release chondrocytes.

The samples may be used as is or the cartilage and/or chondrocytes can be grown in culture using, for example, DMEM (Sigma Cat D5671) supplemented with 10% FCS and 2 mM glutamine.

Chondrocyte cells in culture can either be used as is or treated with known chondrocyte activators—such as IL-1β and TNF-α.

Total RNA may be extracted from the sample using various methods in the art—such as using TRIzol® reagent (Invitrogen) or Rneasy kits (Qiagen). Total RNA may be used as is or complementary DNA may be prepared using, for example, Superscript first Strand cDNA synthesis kits (Invitrogen).

### Assay Method

In one aspect, the present invention relates to an assay method for identifying an agent that modulates chondrocyte arginine transport.

The assay method may be used to identify an agent that is an agonist of CAT-2 that potentiates, enhances or increases the ability of CAT-2 to transport arginine in chondrocytes. The assay method may be used to identify an agent that is an agonist of CAT-2 that potentiates, enhances or increases stimulated chondrocyte iNOS substrate delivery. The agonists may be, for example, natural or modified substrates, ligands, receptors or enzymes or structural or functional mimetics thereof. For example, a chondrocyte expressing CAT-2 may be contacted with an agent. The ability of the agent to modulate CAT-2 activity and/or expression following addition of the agent is then measured.

Preferably, the assay method may be used to identify an agent—such as one or more agents—that is an antagonist of CAT-2 that decreases, reduces or diminishes the ability of CAT-2 to transport arginine in chondrocytes. The assay method may also be used to identify an agent that is an antagonist of CAT-2 that decreases, reduces or diminishes stimulated chondrocyte iNOS substrate delivery. The antagonists may be, for example, natural or modified substrates, ligands, receptors or enzymes or structural or functional mimetics thereof. For example, a chondrocyte expressing CAT-2 may be contacted with an agent. The ability of the agent to modulate CAT-2 activity and/or expression following addition of the agent is then measured.

In the first step of the assay method described herein, agents are identified that modulate the activity and/or expression of CAT-2.

Fusion proteins comprising CAT-2 may be used for high-throughput screening assays to identify modulators of CAT-2 (see D. Bennett et al., J Mol Recognition, 8: 52-58 (1995); and K. Johanson et al., J Biol Chem, 270(16): 9459-9471 (1995)). Another technique for screening for agents that modulate CAT-2 expression and/or activity provides for high throughput screening (HTS) of agents having suitable binding affinity and is based upon the method described in detail in WO 84/03564. For a general reference on screening, see the Handbook of Drug Screening, edited by Ramakrishna Seethala, Prabhavathi B. Fernandes. New York, N.Y., Marcel Dekker, 2001 (ISBN 0-8247-0562-9). The screening method may measure the binding of an agent to CAT-2 by means of a label directly or indirectly associated with the agent. Alternatively, the screening method may involve competition with a labelled competitor.

A plurality of agents may be screened using the methods described below. In particular, these methods may be suited for identifying one or more agents that modulate CAT-2 expression and/or activity and for screening libraries of agents.

Where the candidate compounds are proteins e.g. antibodies or peptides, libraries of candidate compounds may be screened using phage display techniques. Phage display is a protocol of molecular screening, which utilises recombinant bacteriophage. The technology involves transforming bacteriophage with a gene that encodes the library of candidate compounds, such that each phage or phagemid expresses a particular candidate compound. The transformed bacteriophage (which preferably is tethered to a solid support) expresses the appropriate candidate compound and displays it on their phage coat. Specific candidate compounds which are capable of interacting with CAT-2 are enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of compounds utilises eukaryotic or prokaryotic host cells, which are stably transformed with recombinant DNA molecules expressing the library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses. Competitive assays are particularly useful, where the cells expressing the library of compounds are incubated with a labelled antibody, such as ¹²⁵I-antibody, and a test sample such as a candidate compound whose binding affinity to the binding composition is being measured. The bound and free-labelled binding partners are then separated to assess the degree of binding. The amount of test sample bound is inversely proportional to the amount of labelled antibody bound.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Another technique for candidate compound screening involves an approach, which provides high throughput screening for new compounds having suitable binding affinity and is described in detail in WO 84/03564. First, large numbers of different small peptide agents are synthesised on a solid substrate, e.g., plastic pins or some other appropriate surface. Then all the pins are reacted with solubilised protein and washed. The next step involves detecting bound protein. Detection may be accomplished using a monoclonal antibody. Compounds which interact specifically with the protein may thus be identified.

Rational design of candidate compounds likely to be able to interact with CAT-2 may be based upon structural studies of the molecular shapes of the protein and/or its in vivo binding partners. One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

In accordance with the assay method described herein, chondrocyte arginine transport is measured. A person skilled in the art will recognise that there are various ways in which arginine transport in chondrocytes can be measured. Radioactively labelled arginine—such as [³H] arginine—can be used in which the rate of influx into the chondrocyte over a given time internal is measured. Such a method is described in Clinical Science (2002) 102, 645-650. Briefly, an aliquot of cells is added to a solution—such as Ringer's solution—containing radioactively labelled arginine. Aliquots of the cell amino acid mixture are removed at various time intervals—such as 1, 2 and 3 minutes—and the radioactivity in the sample is measured using a liquid scintillation counter. Another method to measure chondrocyte arginine transport is to measure the membrane potential of chondrocytes. Various methods for measuring membrane potential will be familiar to a skilled person. One convenient method is to use dye indicators of membrane potential which have been available for many years and have been widely employed to study cell physiology. These potentiometric dyes are typically organic compounds whose spectral properties are sensitive to changes in membrane potential. They can be classified generally into “fast” dyes, which can follow changes in potential in the millisecond range, and “slow” dyes, which generally operate by a potential-dependent partitioning between the extracellular medium and either the membrane or the cytoplasm. This partitioning of slow dyes occurs by redistribution of the dye via interaction of the voltage potential with ionic charge on the dye. Slow dyes include, but are not limited to, three general chromophore types: cyanines—such as Di-O—C6(3) and Di-S—C2(5), oxonols—such as oxonol-VI and DiS-BaC2(3) and rhodamines—such as rhodamine-123 and TMRE JPW-179 (Loew, Chapter 8 in Biomembrane Electrochemistry, Blank and Vodyanoy, eds., American Chemical Society, Washington, D.C. (1994), pages 151-173.).

In a preferred embodiment of the present invention, the Membrane Potential Blue Assay Kit for FLIPR and FlexStation II (Molecular Devices Corp.) is used to measure membrane potential. The FLIPR Membrane Potential Assay Kit is designed to work in association with many cell types, both adherent and non-adherent. In accordance with the manufacturer's instructions the Loading Buffer is prepared by diluting the vial mixture in 90 mL of 1× Reagent Buffer. An equal volume of Loading Buffer is added to each well. After incubation, the plates are transferred directly to a FLIPR system and the Membrane Potential Assay is started.

It is expected that the assay methods of the present invention will be suitable for both small and large-scale screening of agents as well as in quantitative assays.

In one embodiment of the present invention, medium throughput screening of L-arginine transport in CAT-2B transfected cells measuring radioactively labelled L-arginine transport is used to identify agents that modulate CAT-2 activity and/or expression. By way of example only, cells—such as HEK-293 and CHO-K1 clonal cells lines—transfected with a vector comprising CAT-2B—such as pGEN-IRES-NEO[CAT-2B]—or a control—such as pGEN-IRES-NEO—are plated out in triplicate wells in sterile, clear bottomed plates and incubated overnight. Cells are aspirated, and fresh medium is added and the cells are further incubated. Agents are then added and the plates incubated. Radiolabelled arginine medium (eg. containing 10 μCi [³H] arginine/ml) is added to all wells, and incubated for a pre-determined time between 0 and 60 minutes. The medium is removed and the cells lysed. Arginine uptake in an aliquot of cells is measured in a scintillation counter.

In a further embodiment of the present invention, high throughput screening of L-arginine transport in CAT-2B transfected cells via a membrane potential assay can also be used. For example, cells—such as HEK-293 and CHO-K1 clonal lines—transfected with a vector comprising CAT-2B—such as pGEN-IRES-NEO[CAT-2B]- and a control—such as pGEN-IRES-NEO—are plated out and incubated. Cells are washed and a membrane potential reagent—such as ‘Blue’ membrane potential reagent (Molecular Devices Corp.)—is added to all wells. The plates are further incubated and agents added, followed by further incubation A device for measuring fluorescence—such as a Fluorescence Imaging Plate Reader (FLIPR), or similar kinetic plate reader is used to measure membrane potential changes following the addition of L-arginine. Membrane potential can be measured by monitoring changes in fluorescence, for example, every second for two minutes following the addition of arginine.

CAT-2 expression and/or activity may be detected using various methods known in the art, for example, Northern blotting, in situ hybridisation, nuclease protection assay and PCR.

Northern blotting is a technique for size fractionating RNA in a gel, followed by transfer and immobilisation on a solid support, for example, a membrane in such a manner that the relative positions of the RNA molecules are maintained. The resulting Northern blot is then hybridised with a labelled probe complementary to the mRNA of interest. Signal generated from detection of the probe can be used to determine the size and abundance of the target RNA. Northern blotting analysis may be performed using total RNA and a radioactively labelled CAT-2 probe. The steps involved in Northern analysis include: RNA isolation (total or poly(A) RNA), probe generation, denaturing agarose gel electrophoresis, transfer to solid support and immobilization, prehybridisation and hybridization with probe, washing, detection and finally stripping and reprobing (optional). Kits for practising Northern blotting are commercially available eg. the NorthernMax Kit (Ambion).

In situ hybridisation (ISH) may also be used. Various probes may be used in this method—such as RNA probes, which may be generated and labelled by in vitro transcription procedures or synthetic oligonucleotide probes which may be prepared using various methods known in the art e.g. by automated chemical synthesis. Oligonucleotide probes are labelled during synthesis or by addition of reporter molecules at the 5′ or 3′ end after synthesis. Probes may be labelled in various ways, using radioactive labels e.g. ³²P, or non-radicoactibe labels e.g. DIG. The ISH method comprises a number of steps: (i) Prehybridisation—Samples e.g. sections of tissues—such as cartilage—to be hybridised are incubated with phosphate buffered saline (PBS), washed and then permeabilised with buffer containing either RNase-free Proteinase K or RNase-free Proteinase K. Samples are post-fixed with PBS containing para-formaldehyde and washed with PBS. Samples are acetylated with TEA buffer containing acetic anhydride and incubated with prehybridisation buffer. (ii) In situ hybridisation—A hybridisation buffer is prepared and slides are dipped briefly in distilled water. Prehybridisation buffer is drained from the slides and overlayed with the hybridisation buffer containing labelled probe. Samples are covered and incubated overnight in a humid chamber. (iii) Posthybridisation—Coverslips are removed and samples washed. Unbound RNA probe is removed and the samples again washed. (iv) Immunological detection—Samples are washed with buffer and covered with blocking solution, followed by incubation in a humid chamber with a suitable dilution of labelled antibody—such as sheep anti-DIG-alkaline phosphatase. Samples are again washed and a colour solution prepared containing, for example, nitroblue tetrazolium solution, 5-bromo-4-chloro-3-indolyl-phosphate and levamisole. Each sample is covered with the colour solution and slides are incubated in a humid chamber in the dark. When colour development is optimal, the color reaction is stopped and slides are dipped briefly in distilled water. Samples are counterstained and washed. Additional information concerning in situ hybridisation is available in Komminoth (1992) Diagn. Mol. Pathol. 1, 142-150, Komminoth et al. (1992) Histochemistry 98, 217-228 and Sambrook et al. Molecular Cloning, A Laboratory Manual (1989).

Still another method for determining the activity and/or expression of CAT-2 is the Nuclease Protection Assay, which may be used to detect, and quantify specific CAT-2 mRNA species. A radioactive or non-radioactively labelled single-stranded DNA (or RNA) probe is hybridised to the CAT-2 target mRNA. S1 nuclease is then used to digest all single-stranded RNA and non-duplexed probe. Under optimised conditions, S1 nuclease is highly specific for degrading single-stranded DNA and RNA while exhibiting low activity directed toward DNA or RNA in a DNA:RNA (or RNA:RNA) duplex. The remaining double-stranded hybrid is recovered by ethanol precipitation, separated by gel electrophoresis and visualised by autoradiography. Kits for performing the Nuclease Protection Assay are commercially available—such as the S1-Assay (Ambion).

Yet another method is RT-PCR which is both a sensitive and versatile method that can be used to determine the activity and/or expression of CAT-2. Since RNA cannot serve as a template for PCR, reverse transcription is combined with PCR to make complementary DNA (cDNA) suitable for PCR. This combination of both technologies is referred to as RT-PCR. The technique can be used to, amongst other things, determine the presence or absence of a transcript and to estimate expression levels. Various types of primers may be used for reverse transcription—such as oligo (dT)12-18 which binds to the poly(A) tail at the 3′ end of mammalian RNA; Random hexanucleotides which bind to mRNA at any complementary site and may be better for overcoming difficulties caused by template secondary structure; and specific oligonucleotide primers can be used to selectively prime the CAT-2 RNA sequence of interest. Selection of an appropriate primer for reverse transcription is dependent upon the size and secondary structure of the CAT-2 mRNA.

Preferably, sequence specific primers that anneal only to defined sequences in particular RNAs—such as CAT-2 mRNAs—rather than to the entire RNA population in the sample (e.g., random hexamers or oligo(dT)) are used.

RT-PCR may be one-tube or two-tube. In one-tube RT-PCR, both reverse transcription and PCR are performed in the same tube. A thermostable DNA polymerase—such as Taq DNA polymerase—and all necessary primers and reagents are added during the reaction set-up. cDNA is synthesised by reverse transcription at 37° C. while the DNA polymerase in the reaction mix has little activity. After allowing sufficient time for reverse transcription, the temperature is raised, inactivating the reverse transcriptase and allowing the DNA polymerase to amplify the cDNA. In two-tube RT-PCR, cDNA is first synthesised by reverse transcription. An aliquot of the finished reverse-transcription reaction is then used for PCR. Kits for performing RT-PCR are commercially available—such as the Polymerase One-Step RT-PCR System (Roche Molecular Biochemicals), Titan One-Tube RT-PCR Kit (Roche Molecular Biochemicals) and OneStep RT-PCR Kit (Qiagen).

In a preferred embodiment, CAT-2 expression and/or activity is detected using PCR, preferably, quantitative Real Time PCR analysis (qRT-PCR) on total RNA or cDNA. A variety of qRT-PCR primers can be designed to detect CAT-2 isoforms, and examples are set forth in Table 4.

It will be understood by a skilled person that other methods can be used to determine the activity and/or expression of CAT-2.

Typically, the difference between arginine transport in the absence of the agent and the presence of the agent that is indicative that the agent can modulate chondrocyte arginine transport will be a decrease in arginine transport in the presence of the agent.

Preferably, the assay method is used to screen for agents that are useful in the treatment and/or prevention of diseases.

### Modulate

In relation to chondrocyte arginine transport or the activity and expression of CAT-2, the term “modulate” may refer to preventing, suppressing, reducing, alleviating, restorating, elevating, increasing or otherwise affecting chondrocyte arginine transport and/or the activity and/or expression of CAT-2.

Preferably, the term refers to preventing, suppressing or reducing chondrocyte arginine transport and/or the activity and/or expression of CAT-2.

Thus, the present invention relates to assay methods, processes, and agents that modulate chondrocyte arginine transport and/or the activity and/or expression of CAT-2. Preferably, the assay methods, processes, and agents prevent, suppress or reduce chondrocyte arginine transport and/or the activity and/or expression of CAT-2.

By way of example only, agents that affect chondrocyte arginine transport and/or the activity and/or expression of CAT-2 may bind to the nucleotide sequence encoding CAT-2, or control regions associated with the nucleotide coding sequence, or its corresponding RNA transcript to modify (eg. decrease) the rate of transcription or translation of CAT-2.

Other methods may also be employed, so long as their affect is to modulate chondrocyte arginine transport and/or the activity and/or expression of CAT-2. Such methods may include modulation of expression, activity or degradation of any element, which ultimately results in the modulation of chondrocyte arginine transport and/or the activity and/or expression of CAT-2. The expression of CAT-2 may be modulated, using, for example, antisense oligonucleotides to an mRNA encoding CAT-2 or siRNA. The expression of CAT-2 may also be modulated by modulating the transcription of such an mRNA, or by modulating mRNA processing etc.

Translation of CAT-2 from CAT-2 mRNA may also be regulated as a means of modulating the expression of this protein. Such modulation may make use of agents that are inhibitors of transcription or translation.

Such agents may even modulate the activity of a further entity.

The agents that modulate chondrocyte arginine transport and/or the activity and/or expression of CAT-2 may be used for manufacturing pharmaceutical compositions, which may be used in medicine, in particular for the treatment and/or prevention of diseases—such as inflammatory diseases.

### Agent

The agent according to the present invention may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule, or an antibody, for example, a polyclonal antibody, a monoclonal antibody or a monoclonal humanised antibody.

Various strategies have been developed to produce monoclonal antibodies with human character, which bypasses the need for an antibody-producing human cell line. For example, useful mouse monoclonal antibodies have been “humanised” by linking rodent variable regions and human constant regions (Winter, G. and Milstein, C. (1991) Nature 349, 293-299). This reduces the human anti-mouse immunogenicity of the antibody but residual immunogenicity is retained by virtue of the foreign V-region framework. Moreover, the antigen-binding specificity is essentially that of the murine donor. CDR-grafting and framework manipulation (EP 0239400) has improved and refined antibody manipulation to the point where it is possible to produce humanised murine antibodies which are acceptable for therapeutic use in humans. Humanised antibodies may be obtained using other methods well known in the art (for example as described in U.S. Pat. No. 239,400).

The agent may even be improved analogues of agents that modulate a disease as described herein.

The agents may be attached to an entity (e.g. an organic molecule) by a linker which may be a hydrolysable bifunctional linker.

The entity may be designed or obtained from a library of compounds, which may comprise peptides, as well as other compounds, such as small organic molecules.

By way of example, the entity may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

Typically, the entity will be an organic compound. For some instances, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the entity comprises at least one cyclic group. The cyclic group may be a polycyclic group, such as a non-fused polycyclic group. For some applications, the entity comprises at least the one of said cyclic groups linked to another hydrocarbyl group.

The entity may contain halo groups—such as fluoro, chloro, bromo or iodo groups.

The entity may contain one or more of alkyl, alkoxy, alkenyl, alkylene and alkenylene groups—which may be unbranched- or branched-chain.

The agent may be or may comprise siRNA.

siRNA is the basis of the so called “RNA induced interference” (RNAi) concept, which is a method of post-transcriptional gene regulation that is conserved throughout many eukaryotic organisms.

RNAi is induced by short (typically less than 30 nucleotides) double stranded RNA molecules which are present in the cell (Fire A et al. (1998), Nature 391: 806-811). These short dsRNA molecules (or siRNA) cause the destruction of messenger RNAs which share sequence homology with the siRNA to within one nucleotide resolution (Elbashir S M et al. (2001), Genes Dev, 15: 188-200). It is believed that the siRNA and the targeted mRNA bind to an RNA-induced silencing complex, which cleaves the targeted mRNA. The siRNA is apparently recycled much like a multiple-turnover enzyme, with 1 siRNA molecule capable of inducing cleavage of approximately 1000 mRNA molecules. siRNA-mediated RNAi degradation of mRNA is therefore highly effective for inhibiting expression of a target gene.

The siRNA described herein may comprise partially purified RNA, substantially pure RNA, synthetic RNA, or recombinantly produced RNA, as well as altered RNA that differs from naturally-occurring RNA by the addition, deletion, substitution and/or modification of one or more nucleotides.

Such alterations can include the addition of non-nucleotide material—such as modified nucleotides—to, for example, the end(s) of the siRNA or to one or more internal nucleotides of the siRNA, including modifications that make the siRNA resistant or even more resistant to nuclease digestion.

A number of different types of modifications are known in the art. These include methylphosphonate and phosphorothioate backbones and/or the addition of acridine or polylysine chains at the 3′ and/or 5′ ends of the molecule. The nucleotide sequences may be modified by any method available in the art. Such modifications may be carried out to enhance the in vivo activity or life span of the siRNA.

One or both strands of the siRNA may comprise a 3′ overhang.

Thus, the siRNA may comprise at least one 3′ overhang of, for example, from 1 to about 6 nucleotides (which includes ribonucleotides or deoxynucleotides) in length. If both strands of the siRNA molecule comprise a 3′ overhang, the length of the overhangs can be the same or different for each strand.

In order to enhance the stability of the siRNA, the 3′ overhangs may be stabilised against degradation. The overhangs may be stabilised by including purine nucleotides—such as adenosine or guanosine nucleotides. Alternatively, substitution of pyrimidine nucleotides by modified analogues may be tolerated and may not affect the efficiency of RNAi degradation.

Typically, the siRNA will be in the form of isolated siRNA comprising short double-stranded RNA from about 17 nucleotides to about 29 nucleotides in length—such as approximately 19-25 contiguous nucleotides in length—that are targeted to a target mRNA. The siRNA comprise a sense RNA strand and a complementary antisense RNA strand annealed together by standard Watson-Crick base-pairing, interactions. The sense strand comprises a nucleic acid sequence which is identical to a target sequence contained within the target mRNA.

As used herein, the term “isolated siRNA” means that the siRNA is altered or removed from the natural state through human intervention. An isolated siRNA can exist in substantially purified form, or can exist in a non-native environment such as, for example, a cell into which the siRNA has been delivered.

The sense and antisense strands of the siRNA can comprise two complementary, single-stranded RNA molecules or can comprise a single molecule in which two complementary portions are base-paired and are covalently linked by a single-stranded harpin.

It is understood that human mRNA may contain target sequences in common with their respective alternative splice forms, cognates or mutants. A single siRNA comprising such a common targeting sequence can therefore induce RNAi-mediated degradation of different RNA types, which contain a common targeting sequence.

A target sequence on the target mRNA may be selected from a given sequence—such as a cDNA sequence—corresponding to the target mRNA, using various methods in the art. For example, the rational design of siRNAs is described in Nat Biotechnol. (2004) 22(3):326-30. siRNAs can be designed based on the following guidelines. Firstly, a sequence of around 21 nucleotides in the target mRNA is identified that begins with an AA dinucleotide. Each AA is recorded and the 3′ adjacent nucleotides are identified as potential siRNA target sites. This is based on the observation by Elbashir et al. (EMBO J. (2001) 20: 6877-6888, Nature (2001) 411: 494-498.2 and Genes & Dev. (2001) 15: 188-200) that siRNAs with 3′ overhanging UU dinucleotides are the most effective. However, siRNAs with other 3′ terminal dinucleotide overhangs have been shown to effectively induce RNAi. Preferably, target sites from among the sequences identified above are then further selected using one or more the following criteria: (i) selecting siRNAs with 30-50% GC content; (ii) avoid stretches of >4 T's or A's in the target sequence; (iii) select siRNA target sites at different positions along the length of the gene sequence; and (iv) eliminate any target sequences with more than 16-17 contiguous base pairs of homology to other coding sequences.

If the selected siRNA sequences does not function for silencing, the following steps may be used. A search may be conducted for sequencing errors in the gene and possible polymorphisms. Studies on the specificity of target recognition by siRNA indicate that a single point mutation located in the paired region of an siRNA duplex is sufficient to abolish target mRNA degradation. A second and/or third target may also be selected and the corresponding siRNA prepared and tested.

Although siRNA silencing is highly effective by selecting a single target in the mRNA, it may be desirable to design and employ two independent siRNA duplexes to control the specificity of the silencing effect.

siRNA may be obtained using a number of techniques known to those of skill in the art. For example, the siRNA may be chemically synthesised using appropriately protected ribonucleoside phosphoramidites and a conventional DNA/RNA synthesiser. The siRNA may be synthesised as two separate, complementary RNA molecules, or as a single RNA molecule with two complementary regions.

Further methods for the design of siRNA may be found at the websites of, for example, QIAGEN, Ambion and Ocimum Biosolutions.

siRNA may be recombinantly produced using methods known in the art. For example, siRNA may be expressed from recombinant circular or linear DNA plasmids using any suitable promoter. The recombinant plasmids of the invention can also comprise inducible or regulatable promoters for expression of the siRNA in a particular tissue or in a particular intracellular environment.

The siRNA expressed from recombinant plasmids can either be isolated from cultured cell expression systems by standard techniques. siRNA may be expressed from a recombinant plasmid either as two separate, complementary RNA molecules, or as a single RNA molecule with two complementary regions.

Selection of plasmids suitable for expressing siRNA of the invention, methods for inserting nucleic acid sequences for expressing the siRNA into the plasmid, and methods of obtaining the siRNA are described in, for example, Science (2002) 296: 550-553; Nat. Biotechnol. (2002) 20: 497-500; Genes Dev. (2002), 16: 948-958; Nat. Biotechnol. (2002) 20: 500-505; and Nat. Biotechnol. (2002) 20: 505-508.

As described above, the agent may be an antibody.

Procedures well known in the art may be used for the production of antibodies to CAT-2. Such antibodies include, but are not limited to, polyclonal, monoclonal, chimeric, single chain, Fab fragments and fragments produced by a Fab expression library. Neutralising antibodies, ie., those which may modulate the biological activity of CAT-2, are especially preferred for diagnostics and therapeutics.

For the production of antibodies, various hosts including goats, rabbits, rats, mice, etc. may be immunised by injection with one or more of the polypeptides described herein or any portion, variant, homologue, fragment or derivative thereof or oligopeptide which retains immunogenic properties. Depending on the host species, various adjuvants may be used to increase immunological response. Such adjuvants include, but are not limited to, Freund's, mineral gels—such as aluminium hydroxide—and surface active substances—such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed.

Preferably, the antibody is a monoclonal antibody.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce inhibitor specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for CAT-2 may also be generated. For example, such fragments include, but are not limited to, the F(ab′)₂ fragments which can be produced by pepsin digestion of the antibody molecule and the Fab fragments which can be generated by reducing the disulphide bridges of the F(ab′)₂ fragments. Alternatively, Fab expression libraries may be constructed to allow rapid and easy identification of monoclonal Fab fragments with the desired specificity (Huse W D et al (1989) Science 256:1275-128 1).

An alternative technique involves screening phage display libraries where, for example the phage express scFv fragments on the surface of their coat with a large variety of complementarity determining regions (CDRs). This technique is well known in the art.

Antibodies may be bound to a solid support and/or packaged into kits in a suitable container along with suitable reagents, controls, instructions and the like.

Preferably, the antibody is a humanised antibody.

Humanised antibodies have been obtained by replacing the constant region of a mouse antibody with human protein, but by also replacing portions of the antibody's variable region with human protein. Generally humanised antibodies are 5-10% mouse and 90-95% human. Humanised antibodies were developed to counter the immune responses seen with murine and chimeric antibodies. Data from humanised antibodies used in clinical trials show that humanised antibodies exhibit minimal or no response of the human immune system against them.

A more sophisticated approach to humanised antibodies involves not only providing human-derived constant regions, but also modifying the variable regions as well. This allows the antibodies to be reshaped as closely as possible to the human form. The variable regions of both heavy and light chains contain three complementarity-determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable regions can be “reshaped” or “humanised” by grafting CDRs derived from non-human antibody on the FRs present in the human antibody to be modified. This approach has been reported in, for example, Cancer Res (1993) 53:851-856, Nature (1988) 332:323-327, Science (1988) 239:1534-1536, Proc Natl Acad Sci USA (1991) 88:4181-4185 and J Immunol (1992) 148:1149-1154.

As described above, the agent may be or may comprise an antisense compound, including antisense RNA and antisense DNA, which are capable of reducing the level of expression of the CAT-2 in the chondrocyte which is exposed to the agent. Preferably, the antisense compounds comprise sequences complementary to the mRNA encoding CAT-2.

Preferably, the antisense compounds are oligomeric antisense compounds, particularly oligonucleotides. The antisense compounds preferably specifically hybridize with one or more nucleic acids encoding CAT-2. As used herein, the term “nucleic acid encoding CAT-2” encompasses DNA encoding CAT-2, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of CAT-2.

Antisense constructs are described in detail in U.S. Pat. No. 6,100,090 (Monia et al), and Neckers et al., 1992, Crit Rev Oncog 3(1-2):175-231.

The agents described herein may specifically modulate CAT-2 activity and/or expression such that the agents have substantially no activity towards to other entities—such as other proteins or nucleic acids.

The agents described herein may have substantially no activity towards iNOS.

The agents described herein may be used in combination with iNOS specific inhibitors for increased efficacy. Thus, in a further aspect, there is described a combination comprising an agent as described herein and am iNOS specific inhibitor. Agents that can be used to modulate the generation of NO and so to prevent NO-mediated inflammation are described in U.S. Pat. No. 5,849,794 and WO 03/095421.

The agents described herein may have significant activity towards iNOS, which could offer increased efficacy.

In a preferred embodiment, the agent is selected from the group consisting of an antisense oligonucleotide, an aptamer, a ribozyme, a triplex, PNA or an antibody.

### Prodrug

It will be appreciated by those skilled in the art that the entity may be derived from a prodrug. Examples of prodrugs include certain protected group(s) which may not possess pharmacological activity as such, but may, in certain instances, be administered (such as orally or parenterally) and thereafter metabolised in the body to form an entity that is pharmacologically active.

Suitable pro-drugs may include, but are not limited to, Doxorubicin, Mitomycin, Phenol Mustard, Methotraxate, Antifolates, Chloramphenicol, Camptothecin, 5-Fluorouracil, Cyanide, Quinine, Dipyridamole and Paclitaxel. Agents (e.g. an antibody or a fragment thereof) identified using the methods of the present invention may be chemically linked to an enzyme of interest. Alternatively, the conjugate can be a fusion protein produced by recombinant DNA techniques with the antibody variable region genes and the gene encoding the enzyme. Preferably, the prodrug should be non-toxic, resistant to the action of endogenous enzymes, and be converted into active drug only by the targeted enzyme. The selective activation of anticancer prodrugs by mAb-enzyme conjugates is reviewed in Senetr & Springer (2001) Advanced Drug Delivery Reviews 53, 247-264.

It will be further appreciated that certain moieties known as “pro-moieties”, for example as described in “Design of Prodrugs” by H. Bundgaard, Elsevier, 1985, may be placed on appropriate functionalities of the agents. Such prodrugs are also included within the scope of the invention.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

The agent of the present invention may be capable of displaying other therapeutic properties.

The agent may be used in combination with one or more other pharmaceutically active agents.

If combinations of active agents are administered, then the combinations of active agents may be administered simultaneously, separately or sequentially.

### Stereo and Geometric Isomers

The entity may exist as stereoisomers and/or geometric isomers—e.g. the entity may possess one or more asymmetric and/or geometric centres and so may exist in two or more stereoisomeric and/or geometric forms. The present invention contemplates the use of all the individual stereoisomers and geometric isomers of those entities, and mixtures thereof.

### Pharmaceutical Salt

The agents of the present invention may be administered in the form of a pharmaceutically acceptable salt.

Pharmaceutically-acceptable salts are well known to those skilled in the art, and for example, include those mentioned by Berge et al, in J. Pharm. Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of the agent and the desired acid or base, as appropriate. The salt may precipitate from solution and be collected by filtration or may be recovered by evaporation of the solvent.

The agent of the present invention may exist in polymorphic form.

The agent of the present invention may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The agent may also include all suitable isotopic variations of the agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof of this invention can generally be prepared by conventional procedures using appropriate isotopic variations of suitable reagents.

### Pharmaceutically Active Salt

The agent may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

### Chemical Synthesis Methods

The agent may be prepared by chemical synthesis techniques.

It will be apparent to those skilled in the art that sensitive functional groups may need to be protected and deprotected during synthesis of a compound of the invention. This may be achieved by conventional techniques, for example, as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example, if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts may be separated and purified by conventional methods.

Separation of diastereomers may be achieved by conventional techniques, e.g. by fractional crystallisation, chromatography or H.P.L.C. of a stereoisomeric mixture of a compound of formula (I) or a suitable salt or derivative thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

The agent or variants, homologues, derivatives, fragments or mimetics thereof may be produced using chemical methods to synthesise the agent in whole or in part. For example, if the agent comprises a peptide, then the peptide can be synthesised by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Synthesis of peptide inhibitor agents (or variants, homologues, derivatives, fragments or mimetics thereof) can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent, may be altered during direct synthesis and/or combined using chemical methods with a sequence from other subunits, or any part thereof, to produce a variant agent.

### Chemical Derivative

The term “derivative” or “derivatised” as used herein includes chemical modification of an agent. Illustrative of such chemical modifications would be replacement of hydrogen by a halo group, an alkyl group, an acyl group or an amino group.

### Chemical Modification

The agent may be a modified agent—such as, but not limited to, a chemically modified agent.

The chemical modification of an agent may either enhance or reduce hydrogen bonding interaction, charge interaction, hydrophobic interaction, Van Der Waals interaction or dipole interaction.

In one aspect, the agent may act as a model (for example, a template) for the development of other compounds.

### Pharmaceutical Compositions

Pharmaceutical compositions of the present invention may comprise a therapeutically effective amount of the agent.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

The agents may be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If the agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

The pharmaceutical composition described herein may also be used in combination with conventional treatments of inflammatory diseases.

The pharmaceutical composition may comprise an agent as described herein and an iNOS specific inhibitor.

### Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intra-articular, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

### Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

### Formulation

The component(s) may be formulated into a pharmaceutical composition, such as by mixing with one or more of a suitable carrier, diluent or excipient, by using techniques that are known in the art.

### Diseases

Aspects of the present invention may be used for the treatment or prevention of diseases—such as inflammatory disease. Preferably, the inflammatory disease is selected from the group consisting of diabetes, artheriosclerosis, inflammatory aortic aneurysm, restenosis, ischemia/reperfusion injury, glomerulonephritis, restenosis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, diabetes, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, septic shock, asthma, COPD, and inflammatory dermatosis.

More preferably, the inflammatory disease is or is associated with an arthritis—such as rheumatoid arthritis, psoratic arthritis, coxarthritis, osteoarthritis, or polyarthritis.

In a highly preferred embodiment, the inflammatory disease is or is associated with osteoarthritis or rheumatoid arthritis.

OA represents failure of adiarthrodial (movable, synovial-lined) joint. In idiopathic (primary) OA, the most common form of the disease, no predisposing factor is apparent. Secondary OA is pathologically indistinguishable from idiopathic OA but is attributable to an underlying cause. OA is the most common of all human joint disorders and is the most prevalent arthritic condition in the United States and around the world. Estimates of OA prevalence based on clinical evaluation in various studies show that more than 90% of the population over the age of 70 has OA. The invention is aimed at novel avenues of therapy and prevention of the disease.

OA is a heterogeneous group of conditions that lead to joint symptoms and signs associated with defective integrity of articular cartilage, in addition to related changes in the underlying bone at the joint margins. OA may be either idiopathic (i.e., primary) or secondary to other medical conditions (inflammatory, biochemical, endocrine-related, metabolic, and anatomic or developmental abnormalities). Age is the most powerful risk factor for OA but major trauma and repetitive joint use are also important risk factors for OA.

Rheumatoid arthritis (RA) is a chronic inflammatory disease that causes pain, swelling, stiffness, and loss of function, primarily of the joints. RA is estimated to affect approximately 1 percent of the world's population, suggesting a complex etiology and pathogenesis. The disease process leading to RA begins in the synovium, the membrane that surrounds a joint creating a protective sac. In healthy individuals, the synovium produces synovial fluid that lubricates, nourishes and protects joint tissues. This clear fluid lubricates and nourishes the cartilage and bones inside the joint capsule. In individuals suffering from RA, the immune system attacks the cells inside synovium. Leukocytes infiltrate from the circulation into the synovium causing continuous abnormal inflammation (i.e., synovitis). Consequently, the synovium becomes inflamed, causing warmth, redness, swelling, and pain. The collagen in the cartilage is gradually destroyed, narrowing the joint space and eventually damaging bone. The inflammation causes erosive bone damage in the affected area. During this process, the cells of the synovium grow and divide abnormally, making the normally thin synovium thick and resulting in a joint that is swollen and puffy to the touch (Paul, Immunology (3d ed., Raven Press, 1993)). It is believed that bone damage begins during the first year or two that a person has the disease. This is one reason why early diagnosis and treatment are important in the management of RA. As the disease progresses, abnormal synovial cells begin to invade and destroy the cartilage and bone within the joint. The surrounding muscles, ligaments, and tendons that support and stabilize the joint become weak and unable to work normally. RA also causes more generalized bone loss that may lead to osteoporosis, making bones fragile and more prone to fracture. All of these effects cause the pain, impairment and deformities associated with RA. Although RA almost always develops in the wrists and knuckles, some patients experience the effects of the disease in places other than the joints. For instance, the knees and the ball of the foot are often affected as well. Often, many joints may be involved, and even the spine can be affected. In about 25% of people with RA, inflammation of small blood vessels can cause rheumatoid nodules, or lumps, under the skin. These are bumps under the skin that often form close to the joints. As the disease progresses, fluid may also accumulate, particularly in the ankles. Many patients with RA also develop anemia, or a decrease in the normal number of red blood cells. Other less prevalent effects include neck pain, dry eyes and dry mouth.

On rare occasions, patients may also develop inflammation of the-blood vessels, the lining of the lungs, or the sac enclosing the heart.

RA has several special features that differentiate it from other types of arthritis. For example, RA generally occurs in a symmetrical pattern-if one knee or hand is involved, the other one is also. The disease often affects the wrist joints and the finger joints closest to the hand. RA usually first affects the small joints of the hands and feet, but may also involve the wrists, elbows, ankles and knees. It can also affect other parts of the body besides the joints. In addition, patients with the disease may have fatigue, occasional fever, and a general sense of not feeling well (malaise). Another distinct feature of RA is the variance between individuals. For some, it lasts only a few months or a year or two and subsides without causing any noticeable damage. Other people have mild or moderate disease, with periods of worsening symptoms (flares) and periods in which they feel better (remissions). In severe cases, the disease is chronically active most of the time, lasting for many years, and leading to serious joint damage and disability.

### Diagnosis

In a further aspect, the present invention relates to a method of diagnosing a disease or syndrome in a subject.

In order to provide a basis for the diagnosis of disease, normal or standard levels or patterns of expression or activity of CAT-2 in a sample comprising a chondrocyte should be established. This may be accomplished by testing the levels or patterns of expression or activity of CAT-2 in a sample from one or more normal subjects—such as normal animal or human subjects. The standard levels or patterns of expression or activity of CAT-2 in the sample may be quantified by comparing it to a dilution series of positive controls where the levels or patterns of expression or activity of CAT-2 are in a known amount. Then, standard values obtained from normal samples may be compared with values obtained from samples from subjects potentially affected by a disorder, syndrome or disease related to fluctuations, preferably, an increase in, the levels or patterns of expression or activity of CAT-2. Deviation between standard and subject values may be used to establish the presence of a disease state.

Suitably, the control sample can be a sample comprising a chondrocyte taken from a “normal” non-diseased donor.

A CAT-2 polynucleotide or an isoform or any part thereof (or a variant, homologue, fragment or derivative thereof), may provide the basis for a diagnostic test. For diagnostic purposes, a CAT-2 polynucleotide sequence or an isoform or any part thereof may be used to detect and quantify CAT-2 gene expression and/or activity in conditions, disorders or diseases in which CAT-2 function may be implicated.

A CAT-2 polynucleotide or isoform or any part thereof may be used for the diagnosis of diseases resulting from expression of CAT-2, typically over-expression of CAT-2. For example, polynucleotide sequences encoding CAT-2 may be used in hybridisation or PCR assays of samples—such as tissues from biopsies or autopsies or biological fluids, to detect abnormalities in CAT-2 expression. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

In a preferred embodiment of the present invention, the diagnostic assay described herein is performed as follows. Tissues and/or cells from a subject—such as a human—are obtained from subjects diagnosed with an inflammatory disease—such as an arthritic disease. Nucleic acid—such as DNA, cDNA or RNA—is extracted from the sample. CAT-2 expression and/or activity is detected using PCR, preferably, quantitative Real Time PCR (qRT-PCR). A variety of PCR primers can be designed to detect CAT-2 or an isoform thereof. Examples of such primers are set forth in Table 4. CAT-2B and CAT-2A primers relate to the major reported CAT-2 splice variant forms CAT-2A and CAT-2B; CAT-2 primers detect all CAT-2 mRNAs ie. they do not discriminate between the A and B slice variants.

The diagnostic assays may even be tailored to evaluate the efficacy of a particular therapeutic treatment regime and may be used in animal studies, in clinical trials, or in monitoring the treatment of an individual patient. In order to provide a basis for the diagnosis of disease, a normal or standard profile for CAT-2 expression and/or activity should be established, as mentioned above. Deviation between standard and subject values establishes the presence of the disease state. If one or more disease states are established, an existing therapeutic agent may be administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

A CAT-2 polypeptide, or variant, homologue, fragment, derivative or isoform thereof may be used to produce anti-CAT-2 antibodies, which can, for example, be used diagnostically to detect and quantify CAT-2 levels in disease states. Such antibodies may be used in solution-based, membrane-based, or tissue-based technologies to detect any disease state or condition related to the expression and/or activity of CAT-2 or expression and/or activity of deletions or a variant, homologue, fragment or derivative thereof.

In still a further aspect, the present invention also relates to a method of diagnosing a disease or syndrome in a subject comprising the steps of: (a) detecting the level or pattern of transport of arginine or a derivative thereof in a chondrocyte; and (b) comparing the level or pattern of transport of arginine with that of a control, wherein a difference between the level or pattern of transport of arginine or a derivative thereof in the sample comprising the chondrocyte and the control is indicative of induced CAT-2 and the presence of a disease or syndrome.

Advantageously, the increase in arginine transport that occurs when the activity and/or expression of CAT-2 is increased in chondrocytes may be detected and used as the basis for a further diagnostic assay to detect diseases—such as OA and RA. As already discussed above, arginine transport may be detected using various methods that are known in the art—such as using radio-labelled arginine and/or measuring changes in membrane potential.

In a preferred embodiment, a nuclear imaging technique—such as Positron Emission Tomography (PET) or Single Photon Emission Computed Tomography—may be used by utilising tracers derived from the arginine substrate and/or its metabolite derivatives. Thus, in a preferred embodiment, the tracer is derived from arginine or a derivative thereof. PET is a nuclear medicine imagine technology which allows the three-dimensional, quantitative determination of the distribution of radioactivity and is becoming an increasingly important tool for the measurement of physiological, biochemical, and pharmacological function at a molecular level, both in healthy and pathological states. PET requires the administration to a subject of a molecule labelled with a positron-emitting nuclide (radiotracer) such as ¹⁵O, ¹³N, ₁₁C and ¹⁸F, which have half-lives of 2, 10, 20, and 110 minutes, respectively. Other suitable tracers are described in U.S. Pat. No. 6,187,284.

Accordingly, radiolabeled compounds comprising arginine or a metabolite derivative thereof may be used to effect a method of monitoring the level of arginine transport and employing a nuclear imaging technique for monitoring the distribution of the compound within the sample or the body of a patient. Nuclear imaging dosing depends on the affinity of the compound with CAT-2, the isotope employed and the specific activity of labeling. Persons ordinarily skilled in the art can easily determine optimum nuclear imaging dosages and dosing methodology.

Preferably, modifications required to generate an arginine derived PET tracer will not substantially preclude its facilitated transport by CAT-2B.

CAT-2 specific antibodies are particularly useful for the diagnosis of the diseases described herein. A variety of protocols for competitive binding or immunoradiometric assays using either polyclonal or monoclonal antibodies with established specificities are well known in the art. Such immunoassays typically involve the formation of complexes between the polypeptides described herein and its specific antibody (or similar receptor-binding molecule) and the measurement of complex formation. A two-site, monoclonal based immunoassay utilising monoclonal antibodies reactive to two non-interfering epitopes on a specific receptor protein is preferred, but a competitive binding assay may also be employed. These assays are described in Maddox D E et al (1983, J Exp Med 158:121 1). The diagnostic assays described herein also include methods utilising the antibody and a label to detect the polypeptide in a sample. The polypeptides and antibodies may be used with or without modification. Frequently, the polypeptides and antibodies will be labelled by joining them, either covalently or noncovalently, with a reporter molecule. A wide variety of reporter molecules are known to those of skill in the art.

Furthermore, antibody-tagged tracers may also be used for imaging diseased tissue—such as anti-CAT-2 antibodies tagged with a radioactive tracer e.g. 111In. (J. Nucl. Med. (2005) 46, 514-519). Fluorochrome tagged arginine derivatives may also be used for imaging diseased tissue in an analogous fashion to previously described MMP substrates (Nature Medicine (2001) 7, 743-748) and cathepsin B (Nature Medicine (2002) 8, 757-760).

### Arginine Derivatives

In a further aspect, the present invention relates to a method of identifying a derivative of arginine that can be transported by CAT-2 into or out of a chondrocyte.

Advantageously, the suitably modified arginine derivatives may be used to generate in-situ imaging probes, as described herein.

The term “derivative” as used herein includes chemical modification of a compound—such as arginine. Illustrative of such chemical modifications would be replacement of hydrogen by a halo group, an alkyl group, an acyl group or an amino group.

The arginine derivative may be either an optically active substance or a racemic mixture. The salts of the arginine derivative which can be employed include inorganic acid salt such as hydrochloride, sulfate, hydrobromide, hydroiodide or phosphate, or organic acid salts such as acetate, citrate, p-toluenesulfonate, a fatty acid salt, succinate, maleate, lactate, tartrate, glutamate, aspartate or pyrrolidonecarboxylate.

Specific examples of the arginine derivative include, but are not limited to, N.sup..alpha.-cocoyl-L-arginine stearyl ester hydrochloride, N.sup..alpha.-lauroyl-L-arginine stearyl ester lactate, N.sup..alpha.-lauroyl-L-arginine palmityl ester succinate, N.sup..alpha.-lauroyl-L-arginine stearyl ester citrate, N.sup..alpha.-myristoyl-DL-arginine stearyl ester sulfate, N.sup..alpha.-palmitoyl-L-arginine lauryl ester hydrochloride, N.sup..alpha.-palmitoyl-DL-arginine palmityl ester malate, N.sup..alpha.-stearoyl-L-arginine stearyl ester phosphate, N.sup..alpha.-stearoyl-L-arginine stearyl ester DL-pyrrolidonecarboxylate, hardened beef tallow fatty acid acyl-L-arginine lauryl ester hydrochloride, N.sup..alpha.-cocoyl-L-arginine palmityl ester glutamate, N.sup..alpha.-L-arginine laurylester aspartate, N.sup..alpha.-octanoyl-L-arginine-myristyl ester hydrochloride, N.sup..alpha.-cocoyl-L-arginine isostearyl ester hydrochloride, and N.sup..alpha.-myristoyl-L-arginine octyldodecyl ester hydrochloride.

### Imaging

In a further aspect, the present invention relates to a method of imaging a chondrocyte. According to this aspect of the present invention, a chondrocyte is contacted with labelled arginine or a derivative thereof and the chondrocyte is imaged.

Suitably, the label will provide a conveniently detectable signal (eg. radioactivity). Treating cells with labels and imaging the cells is well known in the art. Specific examples of radiolabelled arginine include, but are not limited to, 14C-labelled arginine, 125I-labelled arginine, 13C-labelled arginine and 15N-labelled arginine. By way of example only, the use of radiolabelled arginine is described in Int J Hyperthermia (1994) 10(1):79-88 and Biochem J. (1977) 166(1):105-13.

As described herein, a preferred imaging method that can be used in accordance with this aspect of the present invention is Positron Emission Tomography (PET) or Single Photon Emission Computed Tomography.

### Variants/Homologues/Derivatives

The present invention encompasses the use of variants, homologues, derivatives and fragments thereof of inter alia CAT-2, CAT-2A and CAT-2B.

The term “variant” is used to mean a naturally occurring polypeptide or nucleotide sequences which differs from a wild-type sequence.

The term “fragment” indicates that a polypeptide or nucleotide sequence comprises a fraction of a wild-type sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 60%, more preferably at least 70%, more preferably at least 80%, more preferably at least 90%, more preferably at least 95%, of the wild-type sequence. Preferably the fragment encodes a protein that has at least 50%, more preferably at least 60%, more preferably at least 70%, more preferably at least 80%, more preferably at least 90%, more preferably at least 95%, and most preferably 100% of the activity of the wild-type protein.

The term “homologue” means an entity having a certain homology with the subject amino acid sequences and the subject nucleotide sequences. Here, the term “homology” can be equated with “identity”.

In the present context, a homologous sequence is taken to include an amino acid sequence, which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence, which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix—such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution—such as basic for basic, acidic for acidic, polar for polar etc. Non-homologous substitution may also occur i.e. from one class of residue to another or alternatively involving the inclusion of unnatural amino acids—such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids—such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe)—such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups—such as methyl, ethyl or propyl groups—in addition to amino acid spacers—such as glycine or β-alanine residues. A further form of variation involves the presence of one or more amino acid residues in peptoid form will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences may be modified by any method available in the art. Such modifications may be carried out to enhance the in vivo activity or life span of nucleotide sequences useful in the present invention.

The present invention may also involve the use of nucleotide sequences that are complementary to the nucleotide sequences or any derivative, fragment or derivative thereof. If the sequence is complementary to a fragment thereof then that sequence can be used as a probe to identify similar coding sequences in other organisms etc.

### Gene Regulation

The present invention encompasses gene regulation whereby nucleotide sequences coding for CAT-2 are regulated in viva. For example, regulation of expression may be accomplished by administering compounds that bind to the nucleotide coding sequence, or control regions associated with the nucleotide coding sequence for CAT-2, or its corresponding RNA transcript to modify the rate of transcription or translation.

By way of example, a nucleotide sequence encoding CAT-2, may be under the control of an expression regulatory element—such as a promoter or a promoter and enhancer. The enhancer and/or promoter may even be active in particular tissues, such that the nucleotide sequence coding for CAT-2 is preferentially expressed. The enhancer element or other elements conferring regulated expression may be present in multiple copies. Likewise, or in addition, the enhancer and/or promoter may be preferentially active in one or more specific cell types—such as chondrocytes.

The level of expression of the nucleotide sequence coding for CAT-2, may be modulated by manipulating the promoter region. For example, different domains within a promoter region may possess different gene regulatory activities. The roles of these different regions are typically assessed using vector constructs having different variants of the promoter with specific regions deleted (that is, deletion analysis).

### General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press.

The invention will now be further described by way of Examples, which are meant to serve to assist one of ordinary skill in the art in carrying out the invention and are not intended in any way to limit the scope of the invention.

### EXAMPLES

### Example 1

### Expression of CAT2 Isoforms in Tissues

The DNA sequences of CAT-2A and CAT-2B alternate splicing variants have been published. (CAT-2A EMBL:HSU76368, J. Biochem. (1997) 36, 6462-6468; CAT-2B EMBL:BC069648, Proc. Natl. Acad. Sci. U.S.A. (2002) 99, 16899-16903). RNA isolated from a number of tissues by standard methods (using kits provided by Qiagen, Invitrogen) was subjected to quantitative rtPCR by standard procedures (using machines provided by ABI and Strategene to manufacturers protocols) using primers/probes specific for CAT-2A, CAT-2B and generic for CAT-2 (i.e do not distinguish between CAT-2A and CAT-2B). These Primers and probes are shown in Table 1. The Taqman profiles are shown in FIG. 1.

The results demonstrate that CAT-2A and CAT-2B have distinct expression profiles, and that CAT-2B is the more abundant isoform. Furthermore, both splice variant forms show limited expression in normal tissues, with by far the highest expression for CAT-2 and CAT-2B in chondrocyte activator stimulated RA chondrocytes.

### Example 2

### Expression of CAT-2B in Cell Lines

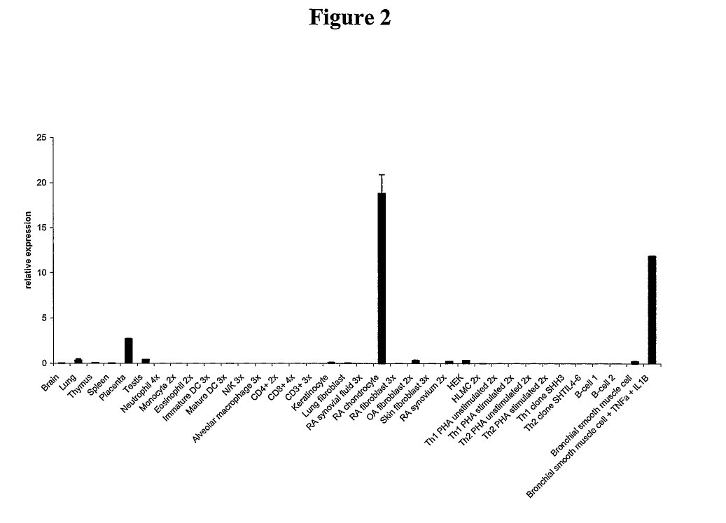

RNA isolated from a number of tissues and cells by standard methods (using kits provided by Qiagen, Invitrogen) was subjected to Taqman quantitative PCR by standard procedures methods (using machines provided by ABI and Strategene to manufacturers protocols) using primers/probes specific for CAT-2B. These Primers and probes are shown in Table 1. The Taqman profiles are shown in FIG. 2.

The results demonstrate that significant CAT-2B expression was limited to chondrocyte activator stimulated RA chondrocytes and TNFα/IL-1β stimulated bronchial smooth muscle cells.

### Example 3

### Inducible Expression of CA T-2 Isoforms in OA Primary Chondrocytes Stimulated with IL-1

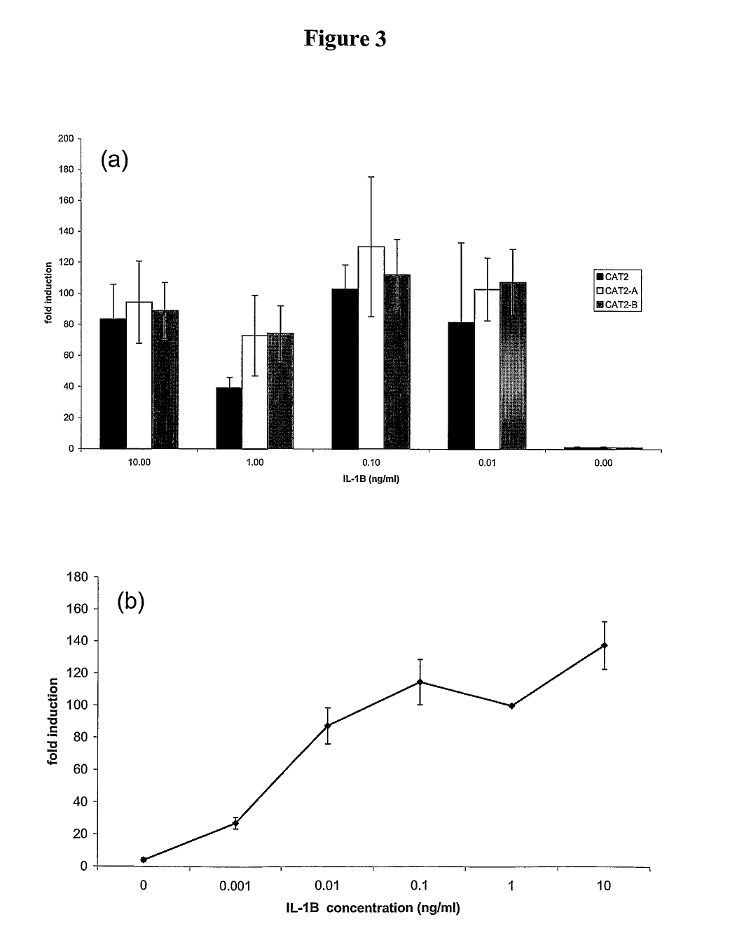

RNA isolated from primary chondrocytes stimulated with a range of IL-1β concentrations was subjected to Taqman quantitative PCR by standard procedures (using machines provided by ABI and Strategene to manufacturers protocols) using primers/probes for: Isoform non-specific CAT-2; specific for CAT-2A isoform or specific for CAT-2B isoform (see FIG. 3(a)). IL-1β dependent CAT-2B induction in OA primary chondrocytes was also taken from five individual OA patents (see FIG. 3(b)). Data are expressed as relative increase in expression (where 1 ng/ml=100%).

The results are shown in FIG. 3.

The results demonstrate that CAT-2A and CAT-2B RNA expression is massively induced in IL-1β stimulated OA primary chondrocytes. Furthermore IL-1β dependent CAT-2B induction is seen in OA primary chondrocytes taken from multiple donors.

### Example 4

### Inducible Expression of CAT-2B in OA Chondrocytes Stimulated with a Range of Chondrocyte Activators

(a) RNA isolated from primary chondrocytes (taken from the joints of two OA patents) stimulated with a range of concentrations of four chondrocyte activators (IL-1β; TNFα; IL-18 & Fibronectin fragments) was subjected to Taqman quantitative PCR by standard procedures (using machines provided by ABI and Strategene to manufacturers protocols) using primers/probes specific for the CAT-2B isoform. (b) Describes the same experiment as (a) where the readout was Taqman analysis of iNOS RNA expression. (c) describes the same experiment as (a) & (b) where the readout is nitrite production.

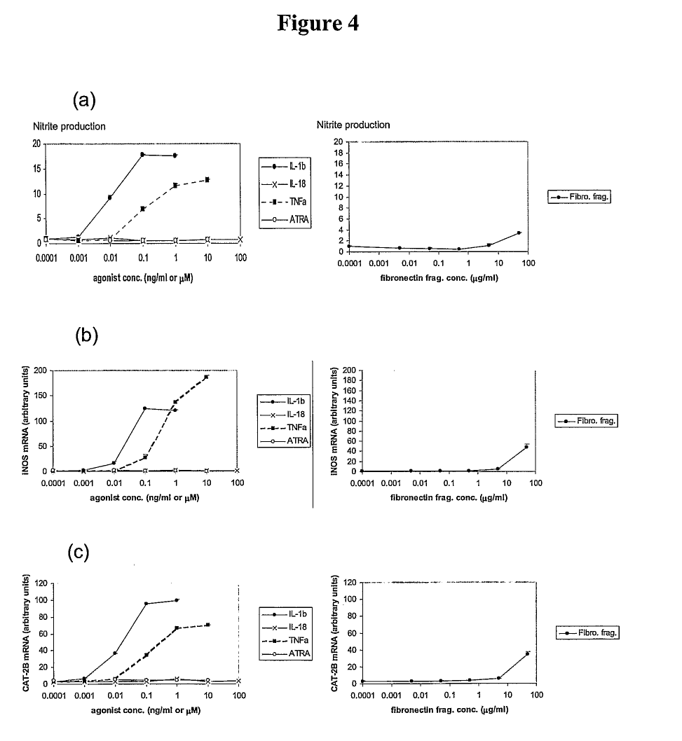

The results are shown in FIG. 4.

The results demonstrate the induction of CAT-2B and iNOS RNA expression in an equivalent fashion and rank potency, in OA primary chondrocytes stimulated with a range of chondrocyte activators including IL-1β, TNFα and Fibronectin fragments. Furthermore, Nitrite production from these variously stimulated OA primary chondrocytes is concomitant with AT-2B and iNOS RNA expression.

### Example 5

### Expression of iNOS and Arginine Transporters in OA Chondrocytes Stimulated with a Range of Chondrocyte Activators

(a) Describes RNA isolated from primary chondrocytes (taken from the joints of an OA patients stimulated with a range of concentrations of four chondrocyte activators (IL-1β; TNFα; IL-18 & Fibronectin fragments) was compared to non treatment control (NTC) samples, and subjected to Taqman quantitative PCR by standard procedures (using machines provided by ABI and Strategene to manufacturers protocols) using primers/probes specific a range of arginine transporters, specifically (a) CAT-1, (b) CAT-2B, (c) y⁺LAT1, (d) B^{(0,+)}AT and the enzyme; (e) iNOS. Two further transporters were investigated (y+LAT2; ATB(0,+)) but expression was below detection. Level of expression was inferred from the maximal critical threshold (CT) value, where <30 CT was deemed high, 30-35 CT low, and >35 CT marginal.

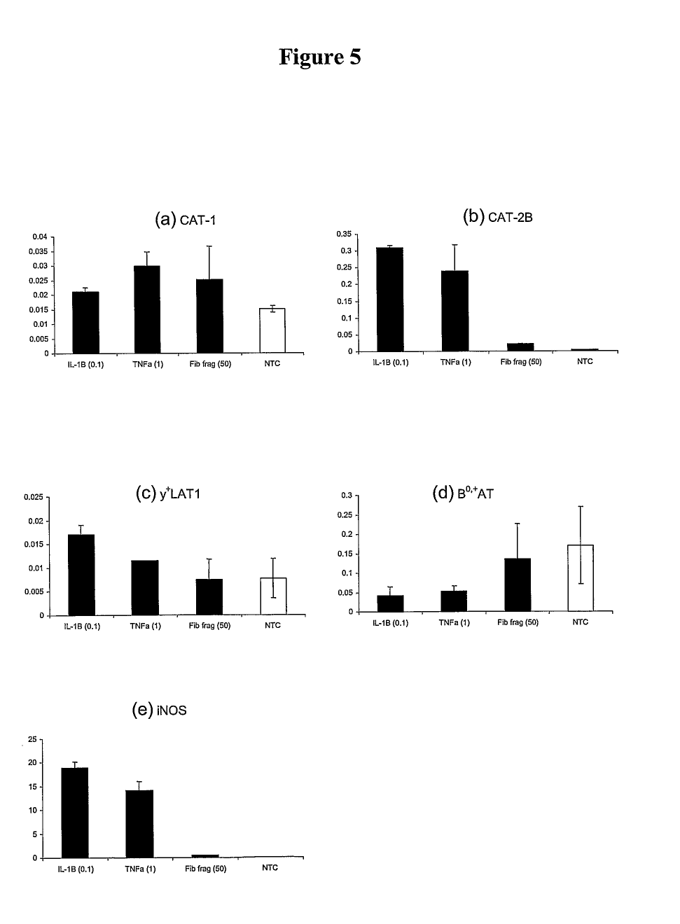

The results are shown in FIG. 5.

This data confirms CAT-2 is the only arginine transporter significantly induced in stimulate OA chondrocytes; and that when induced is probably the most highly expressed arginine transporter in OA chondrocytes.

### Example 6

### Coordinate CAT-2B and iNOS Expression in Cartilage

iNOS and CAT-2B expression were analysed in RNA isolated from articular-cartilage taken from knees of OA patients and Post-Mortem Cadavers.

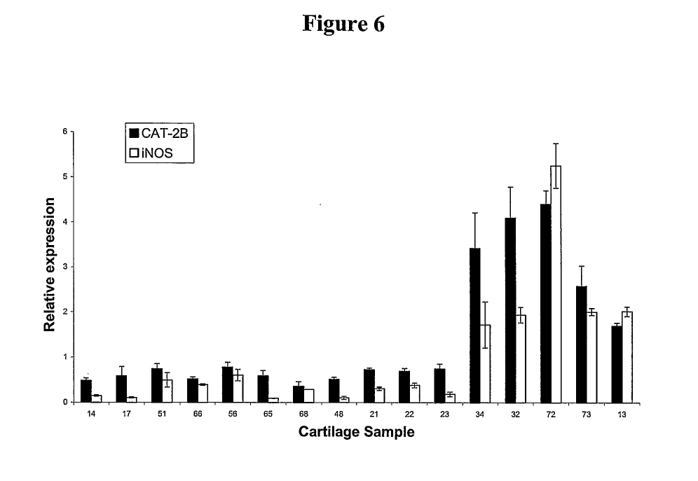

The results are shown in FIG. 6.

The results demonstrate broadly concomitant iNOS and CAT-2B RNA expression in articular-cartilage. Furthermore the level of CAT-2B and iNOS expression was very high and was at least equivalent to that seen in OA primary chondrocytes.

### Example 7

### Coordinate Induction of CAT-2B and iNOS in IL-1β Stimulated OA Primary Chondrocytes, with Arg Transport and NO Readouts

Primary chondrocytes (taken from the joints of two OA patents) were stimulated with 100 pg/ml IL-1β. (a) CAT-2B RNA expression (Taqman); (b) iNOS RNA expression (Taqman); (c) Arginine uptake (radioactive ³H arginine uptake assay) and (d) Nitrite production (Griess Assay) were subsequently determined.

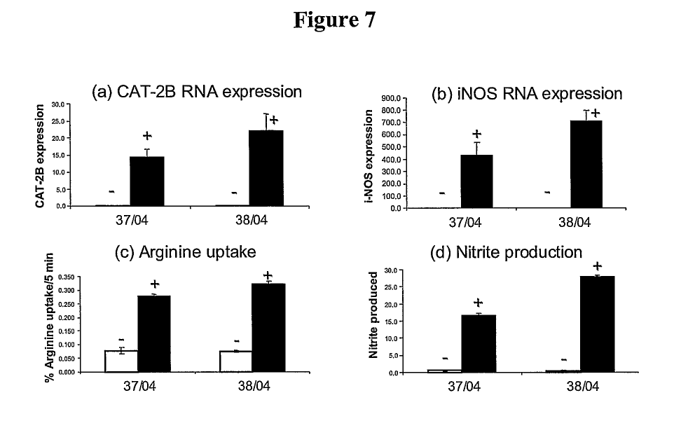

The results are shown in FIG. 7.

The results demonstrate that the induction in CAT-2 expression in cytokine stimulated OA primary chondrocytes is accompanied by a many-fold increase in arginine transport.

### Example 8

### Temporal CAT-2 Knockdown with siRNA Leads to a Reduction in CAT-2B RNA Levels; Arginine Uptake and NO Production in IL-1β Stimulated OA Chondrocytes

### (i) Development of Suitable Transfection Methodology

Optimisation of the window of siRNA delivery to OA chondrocytes in order to obtain maximal mRNA knock-down without lipid-mediated loss of chondrocyte viability was investigated.

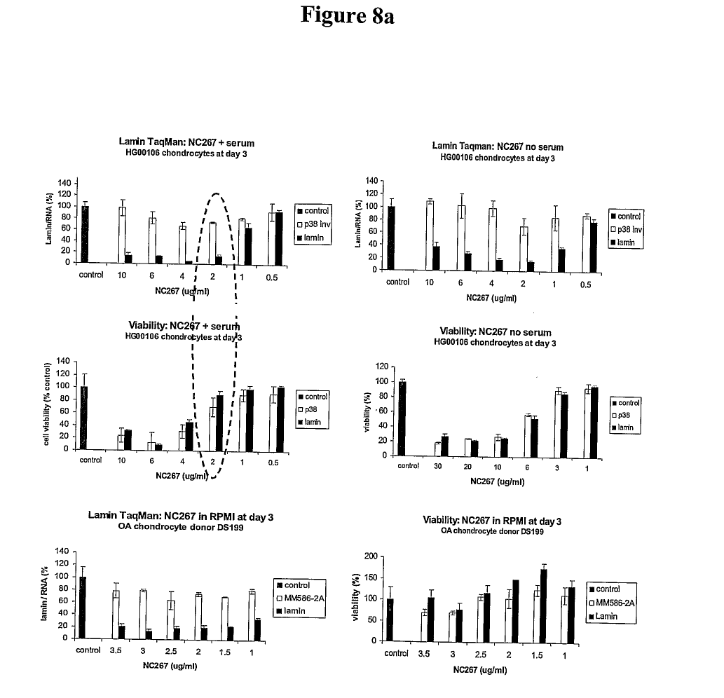

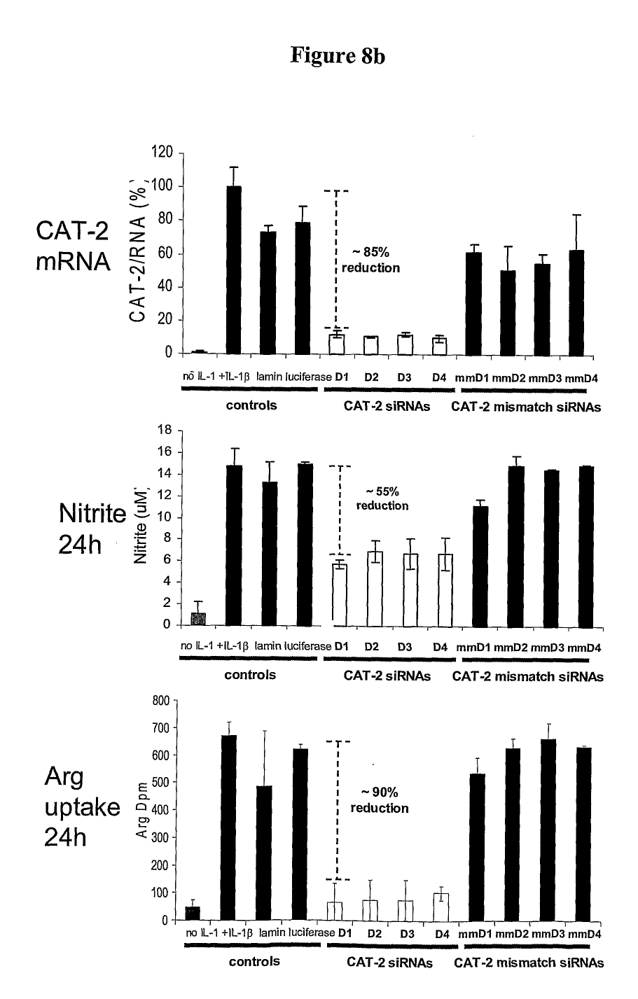

The results are shown in FIG. 8a.

(ii) Screening CAT-2 siRNA Molecules

CAT-2 siRNA molecules from Dharmacon (SMARTpool PLUS) gave efficient knock-down of CAT-2 mRNA, with all four molecules giving up to 80% mRNA reduction (as measured by Taqman using primers and probe to CAT-2). The siRNA sequences are shown in Table 2.

(iii) CAT-2 Knockdown Controls

Mis-match controls (CAT-2 MMsiRNA) for the Dharmacon CAT-2 siRNA molecule, consisting of 4 single base-pair mis-matches per antisense oligo were generated. Control gene mRNAs for lamin or luciferase known to enter the RISC (siRNA processing complex) were also prepared to ensure no global CAT-2 knock-down. The siRNA sequences are shown in Table 3.

(iv) CAT-2 Plays an Important Role in NO Production from Stimulated OA chondrocytes

Experiments in which the effects and or levels of CAT-2 siRNA and controls (CAT-2 MMsiRNA, lamin and luciferase) were examined in IL-1β stimulated chondrocytes, with the multiple readouts of CAT-2 RNA, NO production and arginine transport were performed.

The results are shown in FIG. 8b.

These experiments demonstrate that treatment of IL-1β stimulated OA chondrocytes with CAT-2 siRNAs selectively and significantly reduces CAT-2 RNA expression (by up to 85%); NO production (by up to 55%); and arginine transport (by up to 90%), consolidating our findings that CAT-2 plays an important role in NO production from stimulated OA chondrocytes.

Off target effects, for example knock-down of other Arg transporters, were controlled for by including luciferase and lamin siRNA controls (known to enter RISC) which would control for siRNA-mediated effects. The level of iNOS was not affected by transfection of cells with CAT-2 siRNA molecules (Taqman) and the mRNA for other arginine transporters remained constant (Taqman).

Thus, in conclusion, the siRNA knockdown studies in stimulated OA chondrocytes demonstrate CAT-2B is the main arginine transporter, and plays a major role in NO production from stimulated OA chondrocytes.

### Example 9

### Generation of Mammalian Cell Lines Expressing Cat-2B

The human CAT-2B coding region was isolated from total cDNA generated from IL-1β stimulated human chondrocyte RNA by Polymerase Chain Reaction (PCR). PCR fragments were purified and cloned into pCR®II-TOPO® (Invitrogen), then bi-directionally sequenced prior to subcloning into the AZ proprietory mammalian expression vector pGEN-IRES-NEO (derived from pIRES-neo2 expression vector; BD Biosciences, Palo Alto, Calif., U.S.A.). The mammalian cell lines HEK-293 and CHO-K1 cells were transfected with either the pGEN-IRES-NEO[CAT-2B] or the pGEN-IRES-NEO “empty” plasmids and stably transfected clonal lines were generated under Geneticin (Gibco) selection. HEK-293 cell lines were cultured in “HEK medium”, i.e. DMEM (Sigma Cat D5671) supplemented with 10% FCS and 4 mM glutamine. CHO-K1 cells were cultured in “CHO medium”, i.e. Nut. Mix. F12(HAM) with GlutamaxI supplemented with 10% FCS. Cell lines were incubated at 37° C./5% CO₂. Human CAT-2B protein expression was detected by Western analysis of cell extracts using a commercial anti-human CAT-2 antibody (Orbigen).

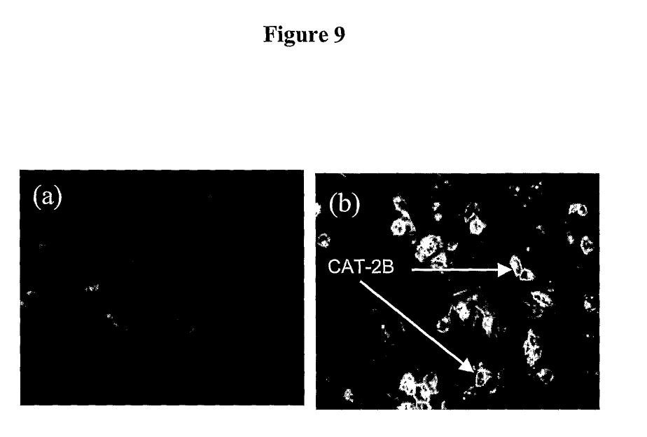

The results are shown in FIG. 9.

The results demonstrate the successful and selective expression of human CAT-2B in pGEN-IRES-NEO[CAT-2B] transfected HEK-293 cells when compared to pGEN-IRES-NEO “empty” plasmid transfected HEK-293 cells.

### Example 10

### Medium Throughput Screening of L-Arginine Transport in CAT-2B Transfected Cells Measuring Radioactively Labelled L-Arginine Transport

HEK-293 and CHO-K1 clonal lines transfected with pGEN-IRES-NEO[CAT-2B] or the pGEN-IRES-NEO are plated out in triplicate wells in sterile, clear bottomed 96 well plates at a density of 50,000 cells/well in 100 μl fresh growth medium and incubated overnight. Cells are aspirated, 100 μl fresh medium added and cells are incubated for a further 15 minutes. Compounds (10 μl) are added to give a final concentration of 10 μM and the plates are incubated for fifteen minutes. 100 μl of radiolabelled arginine medium (containing 10 μCi [³H] Arginine/ml) is added to all wells, and incubated for an appropriate time of between 0 and 20 minutes. The medium is removed and wells rapidly rinsed three times with ice-cold PBS. 150 μl/well 0.5% triton solution in PBS is added and the plate shaken for 1 minute to promote cell lysis. 100 μl from each well is transferred to a 96 well Luma plate (Packard), the plate is incubated at 60° C. for 90 minutes to dry, and [³H] arginine uptake measured in a TopCount (Packard) scintillation counter.

### Example 11

### High Throughput Screening of L-Arginine Transport in CAT-2B Transfected Cells Via Membrane Potential Assay

HEK-293 and CHO-K1 clonal lines transfected with pGEN-IRES-NEO[CAT-2B] or the pGEN-IRES-NEO are plated out in sterile, clear bottomed 384 well plates at a density of 15,000 cells/well and incubated for 18-24 hours at 37° C./5% CO₂. Assay buffer used is 140 mM Choline chloride, 4 mM Potassium chloride, 1 mM magnesium chloride, 2 mM calcium chloride, 20 mM HEPES, pH to 7.4 with KOH. Cells are washed with a 384 well plate washer in assay buffer and 25 μl of ‘Blue’ membrane potential reagent (Molecular Devices Corp.) is added to all wells. The plates are incubated for fifteen minutes at room temperature. Compounds (10 μl) are added to give a final concentration of 10 μM and the plates are incubated for fifteen minutes at room temperature. A Fluorescence Imaging Plate Reader (FLIPR), or similar 384-well kinetic plate reader is used to measure membrane potential changes following the addition of 15 μl/well of L-arginine. The fluorescence is measured using approximately 480 nm excitation and 565 nm emission. Membrane potential is measured by monitoring changes in fluorescence every second for two minutes following the addition of L-arginine.

### Example 12

### A Diagnostic/Prognostic Assay for OA/RA Using qRT-PCR to Identify Induced CAT-2 Isoforms in Human Tissues Including Cartilage

Examples 1-4 demonstrate that CAT-2 transcripts are absent from most tissues, however they are massively induced in stimulated OA chondrocytes, and expressed at a relatively high level in RA chondrocytes: therefore detection of CAT-2 transcripts in joint tissues could be a useful diagnostic/prognostic tool for arthritis treatment. Human tissues and/or cells are obtained from patients diagnosed with an arthritic disease. We describe an example where the tissue is cartilage, although this assay could equally apply to other tissues and cells. Cartilage samples are removed from the joint by a variety of excision methods. The cartilage samples are used as is, or treated with collagenase to break down the cartilage matrix and release chondrocytes. Cartilage and chondrocyte samples are either used as is or grown in culture (DMEM (Sigma Cat D5671) supplemented with 10% FCS and 4 mM glutamine). Cultured cells are either used as is or treated with known chondrocyte activators, examples include IL-1β and TNF-α. Total RNA is extracted from cartilage and chondrocyte samples using TRIzol® reagent (Invitrogen) or Rneasy kits (Qiagen). Total RNA is used as is or complementary DNA is produced using Superscript first Strand cDNA synthesis kits (Invitrogen). CAT-2 expression is detected by quantitative Real Time PCR analysis (qRT-PCR), performed using cartilage or chondrocyte total RNA or cDNA. A variety of qRT-PCR primers can be designed that pick up CAT-2 isoforms, and examples are set forth in Table 4.

CAT-2B and CAT-2A primers relate to the major reported CAT-2 splice variant forms CAT-2A and CAT-2B; CAT-2 primers pick up all CAT-2 mRNAs i.e. do not discriminate between the A and B slice variants.

### Example 13

### Screening for Osteo Arthritis Using CAT-2B Substrates as OA/RA Imaging Probes

Examples 7 and 8 demonstrate that the induction in CAT-2 expression in cytokine stimulated OA primary chondrocytes is accompanied by a many-fold increase in arginine transport. This increase in arginine transport could be detected by Positron Emission Tomography (PET) by utilising PET tracers derived on the CAT-2B substrate arginine and its metabolite derivatives. A requirement is that modifications required to generate an arginine derived PET tracer does not preclude its facilitated transport by CAT-2B. Modified cellular metabolites are commonly used as PET tracers, including derivatives of glucose, adenosine and amino acids and in many instances the native substrate and the modified metabolite have been shown to be transported by the same transport system (Eur J Nucl Med (2001) 29, 754-759; Trends in Neurociences (2005) 28, 117-119). This phenomenon could be used to help in the treatment of inflammatory diseases—such as Osteoarthritis—by providing non-invasive imaging of diseased tissues.

### Example 14

### Arginine Uptake and Nitrite Production Assays on Articular Cartilage

Human diseased joints are obtained from osteoarthritis patients undergoing joint replacement surgery. Articular cartilage is removed and added to 35 ml of sterile filtered 2 mg/ml collagenase (C8051 Sigma Blend collagenase) in chondrocyte medium (i.e. Dulbecco's Modified Eagle's Medium containing 10% (v/v) foetal calf serum, 4 mM Glutamine and 1× Penicillin/Streptomycin solution). The cells are incubated for 24 hours at 37° C. The supernatant containing chondrocytes is strained to remove fragments of undigested cartilage; and the cells are centrifuged (˜10000 g) and washed several times with chondrocyte medium lacking foetal calf serum, then plated in 30 mls chondrocyte medium in T75 tissue culture flasks. The flasks are incubated for approximately three to five days at 37° C. or until the chondrocytes have sufficiently adhered to the flask surface. Cells are then harvested with trypsin and transferred to 96 well tissue culture flasks at approximately 50,000 cells/well in 150 μl 1.1M arginine chondrocyte assay medium (i.e. RPMI-1640 medium containing 4 mM Glutamine and 1× Penicillin/Streptomycin solution) and incubated for between 16-24 hours at 37° C. The medium is then replaced with 150 μl/well 100 μM arginine chondrocyte assay medium, and chondrocyte activator (e.g. 0 pg/ml or 100 pg/ml IL-1β) is added to all wells, and the plates are incubated for approximately 24 hours at 37° C. The medium is then replaced with fresh 150 μl/well 100 μM arginine chondrocyte assay medium, and compounds thought to modulate CAT-2B activity are added to selective wells, then the plates are incubated for approximately 24 hours at 37° C. 50 μl medium is removed to two 96 well plates (one for a nitrite determination assay, one as a backup) and either frozen at −80° C. or room temperature if assayed directly. The remaining medium is aspirated and the cells processed for viability. When transcriptional assays are required, the cell pellets are resuspended in 150 μl RLT buffer (Qiagen RNeasy Lysis Buffer) then stored at −80° C. Arginine uptake (radioactive 3H arginine uptake assay) and Nitrite production (Griess Assay) are subsequently determined to examine the impact of putative CAT-2B modulators on these cellular readouts.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| Oligonucleotide | | Oligonucleotide |
|---|---|---|
| name | Oligonucleotide sequence (5′-3′) | type |
| CAT2-F | CTCCTGTCGCCTTCGTCAG | CAT-2 forward |
| | (SEQ ID No. 5) | primer |
| CAT2-R | TCAGACATCGGGCAAAGGTC | CAT-2 reverse |
| | (SEQ ID No. 6) | primer |
| CAT2-P | AATGATTCCTTGCAGAGCCGCGC | CAT-2 probe |
| | (SEQ ID No. 7) | |
| CAT2A-F | CTTGCCAGAGTGAGTAAGAGGCA | CAT-2A forward |
| | (SEQ ID No. 8) | primer |
| CAT2A-R | GCCATCAAAGCAGAAATGACC | CAT-2A reverse |
| | (SEQ ID No. 9) | primer |
| CAT2A-P | ACCAGTTGCTGCCACGTTGACTGC | CAT-2A probe |
| | (SEQ ID No. 10) | |
| CAT2B-F | TGCTACTTTATCATCGGGTGCA | CAT-2B forward |
| | (SEQ ID No. 11) | primer |
| CAT2B-R | ATCATGTCCACAAGCGCCTT | CAT-2B reverse |
| | (SEQ ID No. 12) | primer |
| CAT2B-P | AAACAGAAAGGCCATCAAAGCTGCCA | CAT-2B probe |
| | (SEQ ID No. 13) | |

TABLE 2

| siRNA | Sequence | |
|---|---|---|
| D1 | GCAATTGGCTTCCTGATTT | (SEQ ID No. 14) |
| D2 | GCAAACAGATTGGTCAGTT | (SEQ ID No. 15) |
| D3 | GTGGCAAACTGGAAGATTA | (SEQ ID No. 16) |
| D4 | GAATTACACTGGTCTTGCA | (SEQ ID No. 17) |

TABLE 3

| siRNA (mis-match | |
|---|---|
| for D1-4 molecules) | Sequence |
| mmD1 | GCAAAUGCCAUCGUGAUUU |
| | (SEQ ID No. 18) |
| mmD2 | GCUAACUGAUAGGUGAGUU |
| | (SEQ ID No. 19) |
| mmD3 | GUCGCAUACUCGAACAUUA |
| | (SEQ ID No. 20) |
| mmD4 | GAUUUAGACUCGUCAUGCA |
| | (SEQ ID No. 21) |

TABLE 4

| Primer Name | Sequence (5′→3′) |
|---|---|
| CAT-2B Forward | TGCTACTTTATCATCGGGTGCA |
| | (SEQ ID No. 22) |
| CAT-2B Reverse | ATCATGTCCACAAGCGCCTT |
| | (SEQ ID No. 23) |
| CAT-2B Probe | AAACAGAAAGGCCATCAAAGCTGCCA |
| | [5′]Fam[3′]DDQ1 |
| | (SEQ ID No. 24) |
| CAT-2A Forward | CTTGCCAGAGTGAGTAAGAGGCA |
| | (SEQ ID No. 25) |
| CAT-2A Reverse | GCCATCAAAGCAGAAATGACC |
| | (SEQ ID No. 26) |
| CAT-2A Probe | ACCAGTTGCTGCCACGTTGACTGC |
| | [5′]Fam[3′]DDQ1 |
| | (SEQ ID No. 27) |
| CAT-2 Forward | CTCCTGTCGCCTTCGTCAG |
| | (SEQ ID No. 28) |
| CAT-2 Reverse | TCAGACATCGGGCAAAGGTC |
| | (SEQ ID No. 29) |
| CAT-2 Probe | AATGATTCCTTGCAGAGCCGCGC |
| | [5′]Fam[3′]DDQ1 |
| | (SEQ ID No. 30) |

1. An assay method for identifying an agent that modulates arginine transport in a chondrocyte comprising the steps of:
(a) identifying an agent that modulates the activity and/or expression of CAT-2; and
(b) measuring arginine transport in the chondrocyte in the presence or absence of said agent,
wherein a difference between: (a) arginine transport in the absence of the agent; and (b) arginine transport in the presence of the agent is indicative that the agent can modulate arginine transport in a chondrocyte.

2. The method according to claim 1, wherein the chondrocyte is selected from the group consisting of a human primary chondrocyte, a passaged primary chondrocyte, an immortalised chondrocyte derived cell line and a synovial fibroblasts.

3. The method according to claim 1, wherein the chondrocyte is isolated or isolatable from an Osteoarthritis (OA) or Rheumatoid Arthritis (RA) patient.

4. The method according to claim 1, wherein the chrondrocyte is stimulated.

5. The method according to claim 4, wherein the chrondrocyte is stimulated by an inflammatory stimulus.

6. The method according to claim 5, wherein the inflammatory stimulus is selected from the group consisting of IL-1α, proIL-1α, IL-1β, TNFα, fibronectin fragment IL-18 and Retinoic Acid.

7. The method according to claim 1, wherein CAT-2 is a CAT-2 isoform.

8. The method according to claim 7, wherein the CAT-2 isoform is or is derived from CAT-2A or CAT-2B.

9. The method according to claim 7, wherein the CAT-2 isoform is expressed in a recombinant cell line.

10. The method according to claim 9, wherein the cell line is HEK-293 or CHO-K1.

11. A method of diagnosing a disease or syndrome in a subject comprising the steps of:
(a) detecting the level or pattern of expression and/or activity of CAT-2 in a chondrocyte; and
(b) comparing the level or pattern of expression and/or activity of CAT-2 with that of a control,
wherein a difference between the level or pattern of expression or activity of CAT-2 in the chondrocyte and the control is indicative of the presence of a disease or syndrome.

12. The method according to claim 11, wherein the disease or syndrome is an inflammatory disease.

13. The method according to claim 12, wherein the inflammatory disease is an arthritic disease.

14. The method according to claim 11, wherein the disease is OA or RA.

15. The method according to claim 11, wherein the chrondrocyte is stimulated.

16. The method according to claim 15, wherein the chrondrocyte is stimulated by an inflammatory stimulus.

17. The method according to claim 16, wherein the inflammatory stimulus is selected from the group consisting IL-1α, proIL-1α, IL-1β, TNFα, fibronectin fragments, IL-18 and Retinoic Acid.

18. A method of diagnosing a disease or syndrome in a subject comprising the steps of:
(a) detecting the level and/or pattern of transport of arginine or a derivative thereof in a chondrocyte; and
(b) comparing—the level and/or pattern of transport of arginine or a derivative thereof with that of an control,
wherein a difference between the level and/or pattern of transport of arginine or derivative thereof in the chondrocyte and the control is indicative of a disease or syndrome.

19. The method according to claim 18, wherein the disease or syndrome is an inflammatory disease.

20. The method according to claim 19, wherein the inflammatory disease is an arthritic disease.

21. The method according to claim 18, wherein the disease is OA or RA.

22. A method of imaging a chondrocyte comprising the steps of:
(a) providing for the uptake of labelled arginine or a derivative thereof into the chondrocyte; and
(b) imaging the chondrocyte.

23. A method of identifying a derivative of arginine that can be transported into and/or out of a chondrocyte by CAT-2 comprising the steps of:
(a) contacting the derivative of arginine with the chondrocyte; and
(b) measuring the transport of the derivative of arginine into and/or out of the chondrocyte.
